(19) United States
(12) Patent Application Publication
(10) Pub. No.: US 2008/0015601 A1
(43) Pub. Date: Jan. 17, 2008

(54) REDUCTION DEVICE AND METHOD OF USE

(76) Inventors: Michael Castro, Uxbridge, MA (US); James Spitler, Taunton, MA (US)

Correspondence Address: CARR LLP (IST), 670 FOUNDERS SQUARE, 900 JACKSON STREET, DALLAS, TX, 75202, US

(21) Appl. No.: 11/762,866

(22) Filed: Jun. 14, 2007

Related U.S. Application Data
(60) Provisional application No. 60/813,548, filed on Jun. 14, 2006.
(60) Provisional application No. 60/825,074, filed on Sep. 8, 2006.
(60) Provisional application No. 60/826,800, filed on Sep. 25, 2006.

(51) Int. Cl. A61F 5/00

(52) U.S. Cl. 606/86

(57) ABSTRACT

A reduction device and a method for using such a device are provided. In one example, the reduction device includes a sleeve, a coupling member configured to couple to a surgical device, and a linkage assembly. The linkage assembly may include multiple links pivotally coupled to the sleeve and coupling member, and a threaded member pivotally coupled to the links. A rotational position of the threaded member may define a position of the sleeve relative to the coupling member.

### CLAIM OF PRIORITY

This application claims priority from U.S. Provisional Patent Application Ser. No. 60/813,548, entitled REDUCTION DEVICE AND METHOD FOR USE, filed on Jun. 14, 2006, U.S. Provisional Patent Application Ser. No. 60/825,074, entitled REDUCTION DEVICE AND METHOD FOR USE, filed on Sep. 8, 2006, and U.S. Provisional Patent Application Ser. No. 60/826,800, entitled REDUCTION DEVICE AND METHOD FOR USE, filed on Sep. 25, 2006, all of which are incorporated herein by reference.

### BACKGROUND

The human spine is a complex structure designed to achieve a myriad of tasks, many of them of a complex kinematic nature. The spinal vertebrae allow the spine to flex in three axes of movement relative to the portion of the spine in motion. These axes include the horizontal (bending either forward/anterior or aft/posterior), roll (bending to either left or right side) and vertical (twisting of the shoulders relative to the pelvis).

In flexing about the horizontal axis into flexion (bending forward or anterior) and extension (bending backward or posterior), vertebrae of the spine must rotate about the horizontal axis to various degrees of rotation. The sum of all such movement about the horizontal axis of produces the overall flexion or extension of the spine. For example, the vertebrae that make up the lumbar region of the human spine move through roughly an arc of 15° relative to its adjacent or neighboring vertebrae. Vertebrae of other regions of the human spine (e.g., the thoracic and cervical regions) have different ranges of movement. Thus, if one were to view the posterior edge of a healthy vertebrae, one would observe that the edge moves through an arc of some degree (e.g., of about 15° in flexion and about 5° in extension if in the lumbar region) centered about a center of rotation. During such rotation, the anterior (front) edges of neighboring vertebrae move closer together, while the posterior edges move farther apart, compressing the anterior of the spine. Similarly, during extension, the posterior edges of neighboring vertebrae move closer together while the anterior edges move farther apart thereby compressing the posterior of the spine. During flexion and extension the vertebrae move in horizontal relationship to each other providing up to 2-3 mm of translation.

In a normal spine, the vertebrae also permit right and left lateral bending. Accordingly, right lateral bending indicates the ability of the spine to bend over to the right by compressing the right portions of the spine and reducing the spacing between the right edges of associated vertebrae. Similarly, left lateral bending indicates the ability of the spine to bend over to the left by compressing the left portions of the spine and reducing the spacing between the left edges of associated vertebrae. The side of the spine opposite that portion compressed is expanded, increasing the spacing between the edges of vertebrae comprising that portion of the spine. For example, the vertebrae that make up the lumbar region of the human spine rotate about an axis of roll, moving through an arc of around 10° relative to its neighbor vertebrae throughout right and left lateral bending.

Rotational movement about a vertical axis relative is also natural in the healthy spine. For example, rotational movement can be described as the clockwise or counter-clockwise twisting rotation of the vertebrae during a golf swing.

In a healthy spine the inter-vertebral spacing between neighboring vertebrae is maintained by a compressible and somewhat elastic disc. The disc serves to allow the spine to move about the various axes of rotation and through the various arcs and movements required for normal mobility. The elasticity of the disc maintains spacing between the vertebrae during flexion and lateral bending of the spine thereby allowing room or clearance for compression of neighboring vertebrae. In addition, the disc allows relative rotation about the vertical axis of neighboring vertebrae allowing twisting of the shoulders relative to the hips and pelvis. A healthy disc further maintains clearance between neighboring vertebrae thereby enabling nerves from the spinal chord to extend out of the spine between neighboring vertebrae without being squeezed or impinged by the vertebrae.

In situations where a disc is not functioning properly, the inter-vertebral disc tends to compress thereby reducing inter-vertebral spacing and exerting pressure on nerves extending from the spinal cord. Various other types of nerve problems may be experienced in the spine, such as exiting nerve root compression in the neural foramen, passing nerve root compression, and ennervated annulus (where nerves grow into a cracked/compromised annulus, causing pain every time the disc/annulus is compressed), as examples. Many medical procedures have been devised to alleviate such nerve compression and the pain that results from nerve pressure. Many of these procedures revolve around attempts to prevent the vertebrae from moving too close to each in order to maintain space for the nerves to exit without being impinged upon by movements of the spine.

Typically, a connector (e.g., a rod) of a spinal stabilization system may be coupled to the head of a screw during a surgical procedure. The head, which may be static or movable, may be designed to receive the rod. For example, the head may include sidewalls that define a groove and the rod may fit into the groove. During the surgical procedure, the rod may be placed into the groove and fastened in place. However, force may need to be applied to reduce the rod (e.g., to move the rod into the groove). Accordingly, an improved reduction device and a method for using such a device are needed.

### SUMMARY

In one embodiment, a device includes a sleeve, a coupling member, and a linkage assembly. The sleeve has a first proximal end, a first distal end configured to receive a rod, and a first longitudinal axis defining a first bore from the first proximal end to the first distal end. The coupling member has a second proximal end, a second distal end configured to couple to a surgical device, and a second longitudinal axis defining a second bore from the second proximal end to the second distal end. The coupling member is positioned at least partially within the sleeve with the first and second longitudinal axes oriented in substantially the same direction. The linkage assembly has a first link pivotally coupled to the sleeve and a first cross member, a second link pivotally coupled to the sleeve and a second cross member, a third link pivotally coupled to the coupling member and the first cross member, a fourth link pivotally coupled to the coupling member and the second cross member, and a threaded member coupling the first and second cross members. A rotational position of the threaded member defines a distance between the first and second cross members.

In another embodiment, a device includes a sleeve, a coupling member, and a threaded member. The sleeve is pivotally coupled to a distal end of first and second links positioned on substantially opposite sides of the sleeve. The coupling member is configured to couple to a surgical device and has a collar pivotally coupled to a distal end of opposing third and fourth links positioned on substantially opposite sides of the coupling member. A longitudinal axis of the coupling member is substantially oriented with a longitudinal axis of the sleeve and the coupling member is rotatable relative to the sleeve and collar. The threaded member is pivotally coupled to a proximal end of the first, second, third, and fourth links. A rotational position of the threaded member defines a position of a distal end of the coupling member relative to a distal end of the sleeve.

In yet another embodiment, a surgical system includes a bone anchor, an extension, and a reduction device. The bone anchor is coupled to a polyaxial head that includes first and second sidewalls forming a groove for receiving a rod. The extension is configured to removably couple to the polyaxial head. The reduction device includes a sleeve, a coupling member, and a linkage assembly. The sleeve is sized to slide over the extension. The coupling member has a distal end positioned at least partially within the sleeve and configured to couple to the extension. The linkage assembly includes a threaded member, first and second links pivotally coupled to the sleeve and the threaded member, and third and fourth links pivotally coupled to the coupling member and the threaded member. A rotational position of the threaded member defines a position of a distal portion of the sleeve relative to the polyaxial head.

In still another embodiment, a method includes inserting a distal end of a bone anchor into a vertebral body and coupling an extension to a polyaxial head connected to a proximal end of the bone anchor. The method also includes sliding a sleeve of a reducing device over the extension and coupling a coupling member of the reducing device to the extension within the sleeve, where the coupling member is coupled to the sleeve via a linkage assembly. The method also includes moving a distal end of first and second links of the linkage assembly away from a distal end of third and fourth links of the linkage assembly by rotating a threaded member of the linkage assembly coupled to the first, second, third, and fourth links, where the moving alters a position of a distal end of the sleeve relative to the polyaxial head.

In another embodiment, a device includes a sleeve, a coupling member, and a linkage assembly. The sleeve has a first proximal end, a first distal end configured to receive a rod, and a first longitudinal axis defining a first bore from the first proximal end to the first distal end. The coupling member has a second proximal end, a second distal end configured to couple to a surgical device, and a second longitudinal axis defining a second bore from the second proximal end to the second distal end. The coupling member is positioned at least partially within the sleeve with the first and second longitudinal axes oriented in substantially the same direction. The linkage assembly has first and second gears rotationally coupled to the sleeve, and first and second arms coupled to the first and second gears, respectively. Each of the first and second gears includes a plurality of teeth that engage a plurality of teeth on the coupling member, and a position of the first and second arms defines a position of the coupling member relative to the sleeve.

In yet another embodiment, a device includes a sleeve, a handle, a threaded boss member, and a driver. The sleeve has a first proximal end with a flange, a first distal end configured to receive a rod, and a first longitudinal axis defining a first bore from the first proximal end to the first distal end. The handle has a second longitudinal axis defining a second bore through the handle, where the second bore includes a first threaded portion and a groove for receiving the flange of the sleeve. The threaded boss member has a third proximal end, a third distal end configured to couple to a surgical device, and a third longitudinal axis defining a third bore from the third proximal end to the third distal end. The third proximal end includes a lip extending around an opening to the third bore, and the coupling member is positioned at least partially within the sleeve. The driver has a fourth proximal end, a fourth distal end, a fourth longitudinal axis oriented in substantially the same direction as the first longitudinal axis, and a flange configured to abut the lip and retain the fourth distal end in the third bore.

### BRIEF DESCRIPTION OF THE DRAWINGS

Aspects of the present disclosure are best understood from the following detailed description when read with the accompanying figures. It is emphasized that various features may not be drawn to scale. In fact, the dimensions of the various features may be arbitrarily increased or reduced for clarity of discussion.

FIG. 1 illustrates one embodiment of a reduction device.

FIG. 2 illustrates one embodiment of a sleeve that may be used in the reduction device of FIG. 1.

FIG. 3 illustrates one embodiment of a reduction handle that may be used in the reduction device of FIG. 1.

FIG. 4 illustrates one embodiment of a threaded boss member that may be used in the reduction device of FIG. 1.

FIG. 5 illustrates one embodiment of a drive transmission that may be used in the reduction device of FIG. 1.

FIG. 6 illustrates a cross-sectional view of one embodiment of the reduction device of FIG. 1.

FIG. 7 illustrates an enlarged upper portion of the cross-sectional view of FIG. 6.

FIG. 8 illustrates one embodiment of the reduction device of FIG. 1 with surgical components.

FIG. 9 illustrates a cross-sectional view of one embodiment of the reduction device of FIG. 8.

FIG. 10a and 10b illustrate an enlarged upper portion and an enlarged lower portion, respectively, of the cross-sectional view of FIG. 9.

FIG. 11 is a flow chart of one embodiment of a method for using the reduction device of FIG. 1.

FIG. 12 illustrates another embodiment of a reduction device with surgical components.

FIG. 13 illustrates a cross-sectional view of one embodiment of the reduction device of FIG. 12.

FIG. 14 illustrates an enlarged upper portion of the cross-sectional view of FIG. 13.

FIG. 15 illustrates one embodiment of a concentric circle structure that may be used within a component of the reduction device of FIG. 12.

FIG. 16 illustrates a perspective view of yet another embodiment of a reduction device.

FIG. 17 illustrates a side view of the reduction device of FIG. 16.

FIG. 18 illustrates a cross-sectional view of the reduction device of FIG. 17 from the opposite side.

FIG. 19 illustrates an enlarged view of an upper portion of the cross-sectional view of FIG. 18.

FIG. 20 illustrates one embodiment of a linkage assembly that may be used in the reduction device of FIG. 16.

FIG. 21 illustrates a side view of one embodiment of a linkage assembly and a threaded member that may be used in the reduction device of FIG. 16.

FIG. 22 illustrates a cross-sectional view of the linkage assembly and threaded member of FIG. 21 from the opposite side.

FIG. 23a illustrates a side view of one embodiment of a portion of the reduction device of FIG. 16.

FIG. 23b illustrates a cross-sectional view of the portion of FIG. 23a.

FIG. 24 illustrates a perspective view of one embodiment of a sleeve that may be used in the reduction device of FIG. 16.

FIGS. 25a and 25b illustrate an upper portion of the reduction device of FIG. 16 in first and second positions, respectively.

FIGS. 26a and 26b illustrate a lower portion of the reduction device of FIG. 16 in first and second positions, respectively.

FIG. 26c illustrates a cross-sectional side view of the reduction device of FIG. 26b.

FIG. 27 is a flow chart of one embodiment of a method for a surgical procedure using the reduction device of FIG. 16.

FIG. 28 illustrates a perspective view of still another embodiment of a reduction device.

FIG. 29 illustrates a cross-sectional side view of the reduction device of FIG. 28.

FIG. 30 illustrates a perspective view of another embodiment of a reduction device.

FIG. 31 illustrates a side view of the reduction device of FIG. 30.

FIG. 32 illustrates a cross-sectional view of the reduction device of FIG. 31.

FIG. 33 illustrates a front view of another embodiment of a reduction device.

FIG. 34 illustrates a side view of one embodiment of a portion of the reduction device of FIG. 33.

FIG. 35 illustrates front view of one embodiment of a portion of the reduction device of FIG. 33.

FIG. 36 illustrates a cross-sectional view of one embodiment of a section of the reduction device of FIG. 35 along lines A-A.

FIG. 37 illustrates one embodiment of a coupling member that may be used in the reduction device of FIG. 33.

FIG. 38 illustrates a view of one embodiment of a lower section of the coupling member of FIG. 37.

FIG. 39 illustrates a perspective view of one embodiment of a lower section of the coupling member of FIG. 37.

FIG. 40 illustrates a cross-sectional view of one embodiment of a lower section of the coupling member of FIG. 37.

FIG. 41 illustrates one embodiment of an extension with a lower section of the coupling member of FIG. 37.

FIG. 42 illustrates one embodiment of a ramp ring with a lower section of the coupling member of FIG. 37.

FIG. 43 illustrates a perspective view of one embodiment of a ramp ring with a lower section of the coupling member of FIG. 37.

FIG. 44 illustrates one embodiment of a ramp ring that may be used with the coupling member of FIG. 37.

FIG. 45 illustrates a cross-sectional view of one embodiment of a lower section of the reduction device of FIG. 33.

FIG. 46 is a flow chart of one embodiment of a method for a surgical procedure using the reduction device of FIG. 33.

### WRITTEN DESCRIPTION

It is to be understood that the following disclosure provides many different embodiments, or examples, for implementing different features of the disclosure. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

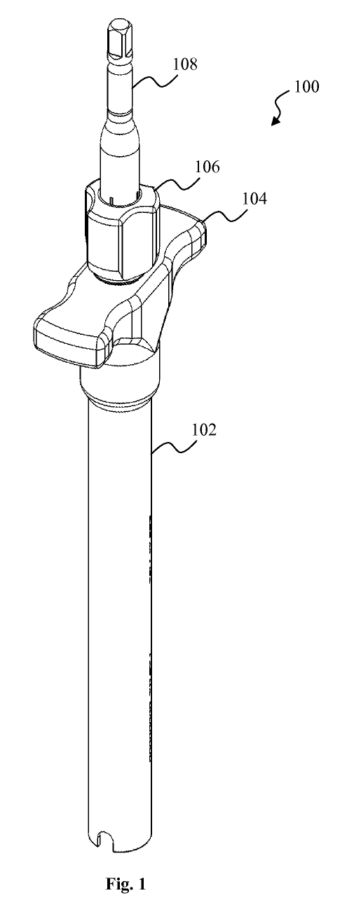

Referring to FIG. 1, in one embodiment, a reduction device 100 is illustrated. The reduction device 100 may include a sleeve 102, a reduction handle 104, a threaded boss member 106, and a drive transmission 108. As will be discussed below in greater detail, the reduction device 100 may be used to apply pressure to seat a rod in a screw head during a spinal surgery procedure. A locking cap may then be tightened, either with all or a portion of the reduction device 100 in place or after the reduction device has been removed, to secure the rod to the screw head. Accordingly, the sleeve 102, reduction handle 104, threaded boss member 106, and drive transmission 108 may interact to provide the force needed to seat the rod while eliminating or minimizing stress to the threads of the locking cap.

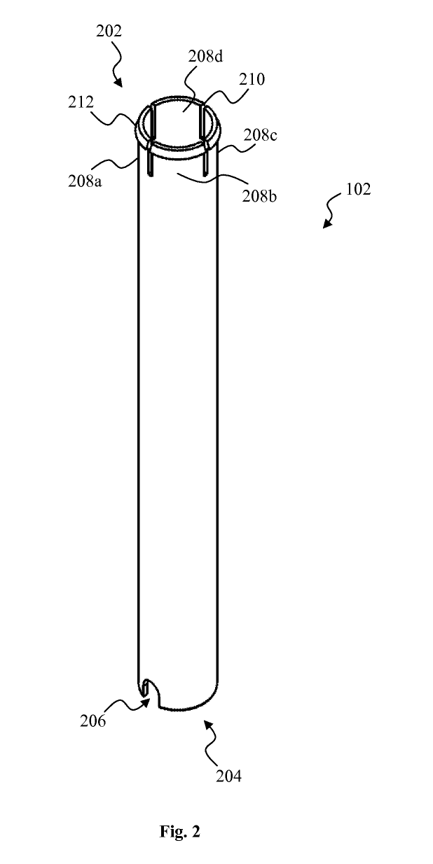

Referring to FIG. 2, one embodiment of the sleeve 102 of FIG. 1 is illustrated. The sleeve 102 may include a proximal end 202 (relative to a user of the reduction device 100) and a distal end 204, and may be relatively cylindrical around a longitudinal axis (not shown) extending from the proximal end to the distal end. The sleeve 102 may include one or more slots 206 formed in the distal end 204 to receive a rod or other surgical device component, as will be discussed later.

In the present example, the proximal end 202 may include multiple sectioned portions (e.g., tabs) 208a, 208b, 208c, and 208d. It is understood that more or fewer sectioned portions may be provided, and that the number of sectioned portions illustrated in FIG. 2 is for purposes of example only. The end of each of the tabs 208a-208d may include a lip 212 extending outwards from the sleeve 102. As will be described later, the tabs 208a-208d and the lip 212 may aid the sleeve 102 in retaining the reduction handle 104 in a manner that allows the reduction handle to rotate relative to the sleeve.

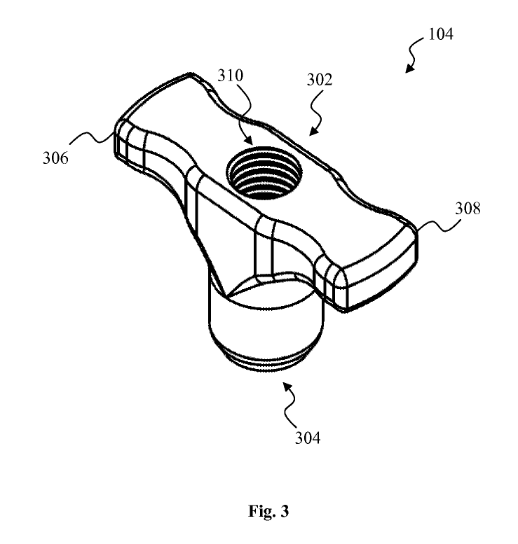

Referring to FIG. 3, one embodiment of the reduction handle 104 of FIG. 1 is illustrated. The reduction handle 104 may include a proximal end 302 (relative to a user of the reduction device 100) and a distal end 304. The proximal end 302 may include a gripping means (illustrated as protrusions 306 and 308) to enable a user of the reduction device 100 to apply pressure, as will be described later. A partially threaded bore 310 may pass through the reduction handle 104. In the present example, an upper portion of the partially threaded bore 310 is sized to accept an outer thread portion of the threaded boss member 106. A lower portion (not shown) of the partially threaded bore 310 may have a larger diameter than the upper portion, and may be smooth with a slot or other indentation to accept the lip 212 of the sleeve 102.

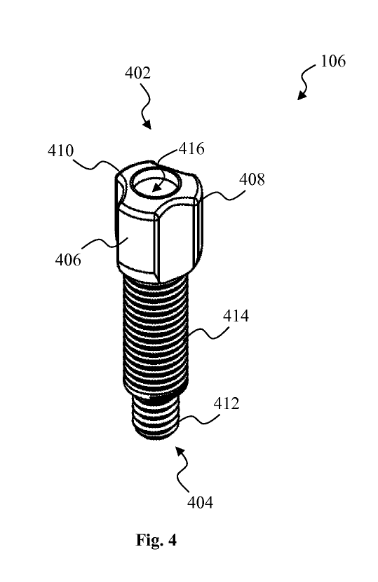

Referring to FIG. 4, one embodiment of the threaded boss member 106 of FIG. 1 is illustrated. The threaded boss member 106 may include a proximal end 402 (relative to a user of the reduction device 100) and a distal end 404. The proximal end 402 may include a gripping means (illustrated as protrusions 406, 408, and 410) to enable a user of the reduction device 100 to apply pressure, as will be described later. In the present example, the threaded boss member 106 may include an inner threaded portion 412 and an outer threaded portion 414. The inner threaded portion 412 may engage an extension, as will be discussed later. The outer threaded portion 414 may engage the upper portion of the partially threaded bore 310 of the reduction handle 104. In some examples, the threads of the outer threaded portion 414 may be tapered. A bore 416, which may be smooth, may extend through the threaded boss member 106 from the proximal end 402 to the distal end 404. As will be described later, the bore 416 may include a wider upper portion (not shown) surrounded by a lip extending inwards (e.g., resulting in the diameter of the bore 416 illustrated in FIG. 4) and may then narrow towards the distal end 404.

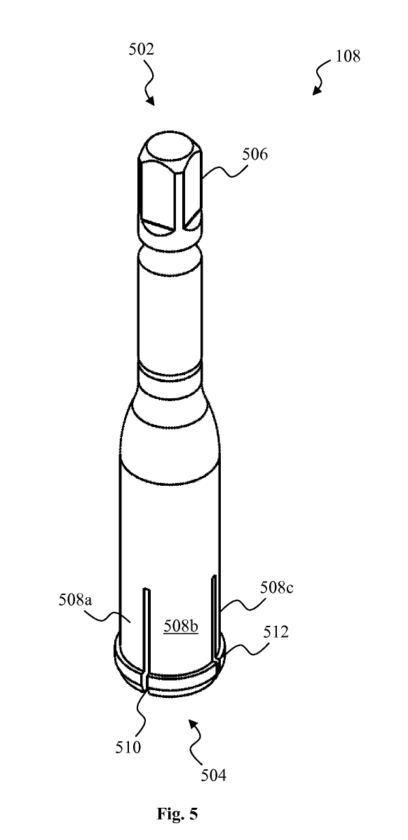

Referring to FIG. 5, one embodiment of the drive transmission 108 of FIG. 1 is illustrated. The drive transmission 108 may include a proximal end 502 (relative to a user of the reduction device 100) and a distal end 504. The proximal end 502 may include a drive surface 506 configured to engage a tool (not shown). The proximal end 502 may be relatively solid, while the distal end 504 may be at least partially hollow.

In the present example, the distal end 504 may include multiple sectioned portions (e.g., tabs) 508a, 508b, 508c, and 508d (not shown). It is understood that more or fewer sectioned portions may be provided, and that the number of sectioned portions illustrated in FIG. 5 is for purposes of example only. The end of each of the tabs 508a-508d may include a lip 512 extending outwards from the drive transmission 108. As will be described later, the tabs 508a-508d and the lip 512 may aid the drive transmission 108 in retaining the threaded boss member 106 in a manner that allows the drive transmission to rotate relative to the threaded boss member.

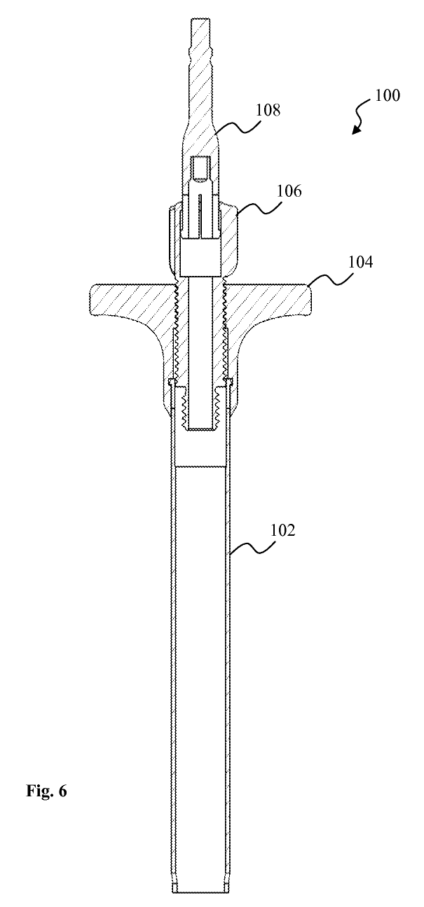

Referring to FIG. 6, a cross-sectional view of one embodiment of the reduction device 100 of FIG. 1 is illustrated. Illustrated are the sleeve 102, reduction handle 104, threaded boss member 106, and drive transmission 108, as well as one possible relationship between the components.

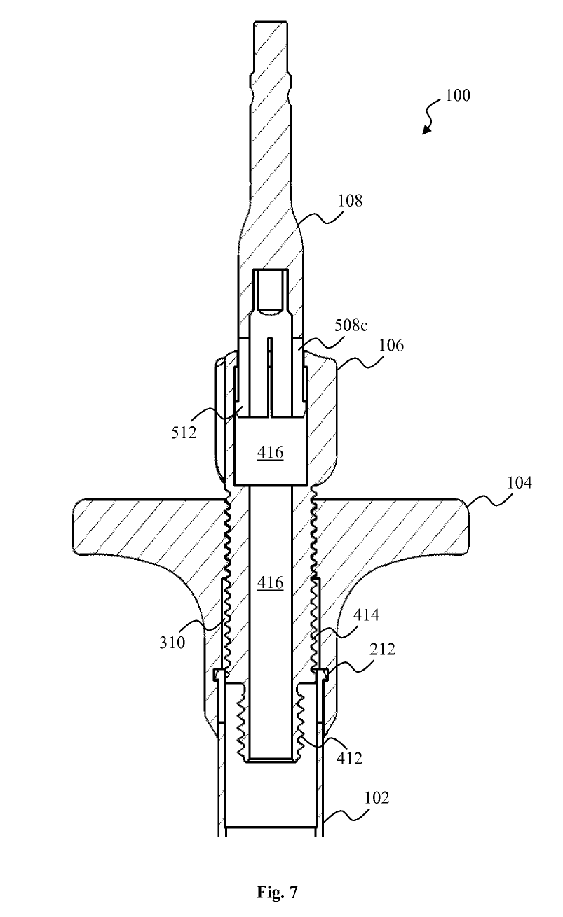

With additional reference to FIG. 7, an enlarged upper portion of the cross-sectional view of FIG. 6 is illustrated. As can be seen, FIG. 7 provides one example of the interaction between the sleeve 102, reduction handle 104, threaded boss member 106, and drive transmission 108.

The sleeve 102 may fit into a lower portion of the bore 310 of the reduction handle 104. When inserted, the tabs 208a-208d may be compressed inwardly until the lip 212 fits into a groove in the lower smooth portion of the bore 310. When this occurs, the tabs 208a-208d may snap outwardly, holding the lip 212 in the groove. In addition, the bore 310 may be slightly narrower above the groove. Accordingly, the sleeve 102 may freely rotate with respect to the reduction handle 104 while still retaining the reduction handle.

The reduction handle 104 may receive the threaded boss member 106 in the bore 310. As illustrated in FIG. 7, the upper portion of the bore 310 may be threaded to engage the outer threaded portion 414 of the threaded boss member 106 and the lower portion of the bore may be wider and smooth. In the present example, the lower portion may have a diameter large enough that the sides of the bore 310 do not contact the crest of the threads of the outer threaded portion 414. The outer threaded portion 414 may be longer than the upper threaded portion of the bore 310 to allow for a relatively large range of controllable motion between the reduction handle 104 and the threaded boss member 106.

The distal end 404 (FIG. 4) of the threaded boss member 106 may engage the reduction handle 104 using the outer threaded portion 414 and may engage an extension (not shown) using the inner threaded portion 412. The proximal end 402 of the threaded boss member 106 may be retained by the drive transmission 108. More specifically, the tabs 508a-508d of the drive transmission 108 may be compressed inwardly and inserted into the bore 416 of the threaded boss member 106. Once past the relatively narrow opening of the bore 416, the tabs 508a-508d may snap outwardly, and the lip 512 may aid in retaining the tabs in the bore. Accordingly, the drive transmission 108 may freely rotate with respect to the threaded boss member 106 while still being retained by the threaded boss member.

As shown, the upper portion of the bore 416 may be wider than the lower portion, and the distal end 504 of the drive transmission 108 may be unable to enter the lower portion. Accordingly, the upper portion of the bore 416 may be defined to provide a certain range of vertical movement of the drive transmission 108 with respect to the threaded boss member 106.

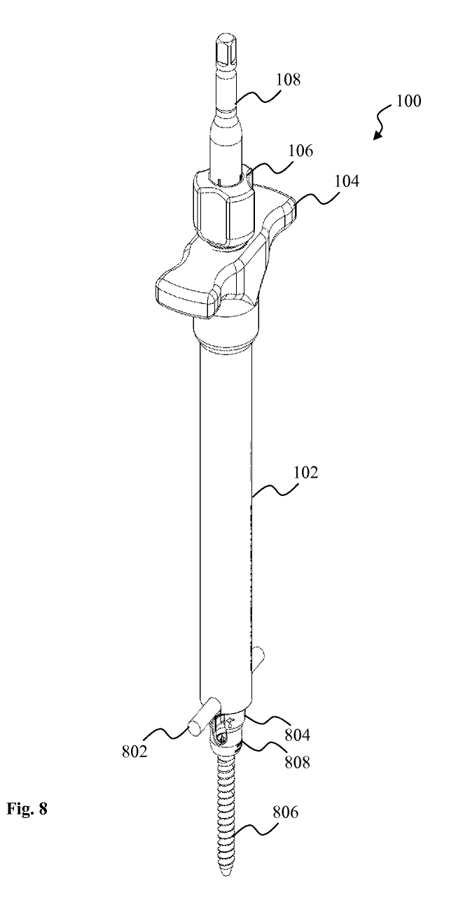

Referring to FIG. 8, one embodiment of the reduction device 100 of FIG. 1 is illustrated with a rod 802. Also illustrated are a portion of an extension 804 and a fastener 806 (e.g., a bone screw) coupled to a polyaxial head 808. Examples of the rod 802, extension 804, fastener 806, and polyaxial head 808 are described in U.S. patent application Ser. No. 10/690,211, filed on Oct. 21, 2003, U.S. patent application Ser. No. 10/990,272, filed on Nov. 16, 2004, and U.S. patent application Ser. No. 10/989,715, filed on Nov. 16, 2004, all of which are hereby incorporated by reference. As described therein, the bone screw 806 may be coupled to the polyaxial head 808, which may in turn be removably coupled to the extension 804. The extension 804 may include a bore that permits the rod 802 and/or a tool or other device to be passed through the extension and to interact with the polyaxial head. As shown in FIG. 8, the rod 802 may not be seated into the polyaxial head 808 as desired. Accordingly, the reduction device 100 may be used to seat the rod 802.

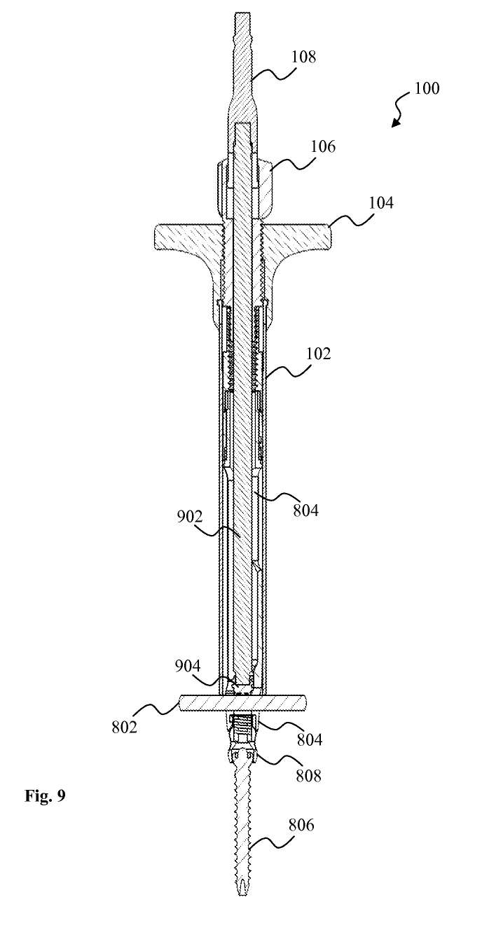

With additional reference to FIG. 9, a cross-sectional view of one embodiment of the reduction device 100 of FIG. 8 is illustrated. Also illustrated in FIG. 9 are a driver 902 and a locking cap 904. The locking cap 904 may be used to secure the rod 802 to the polyaxial head 808 once the rod is seated in the polyaxial head. It is understood that the locking cap 904 may be used to aid in seating the rod 802, but this may place undesirable stress on the threads of the locking cap. In the present example, although the driver 902 engages the drive transmission 108, it may not be coupled to any portion of the reduction instrument 100.

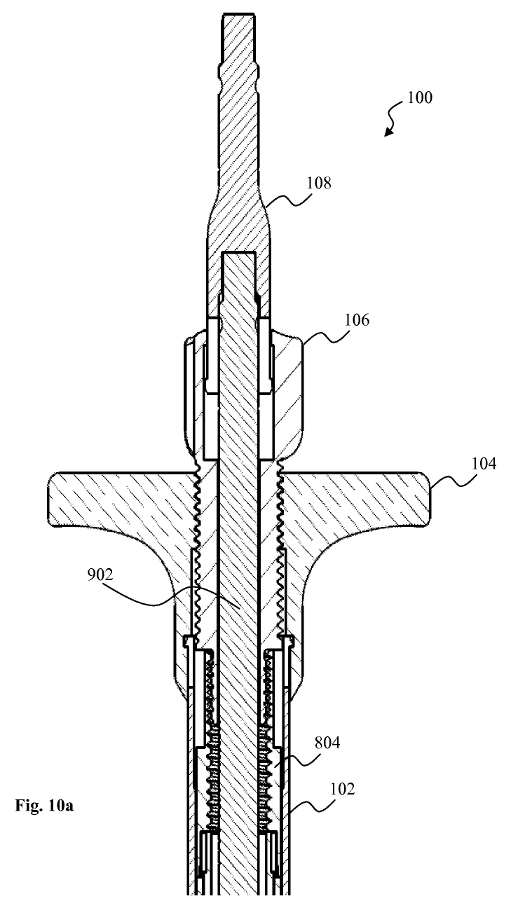

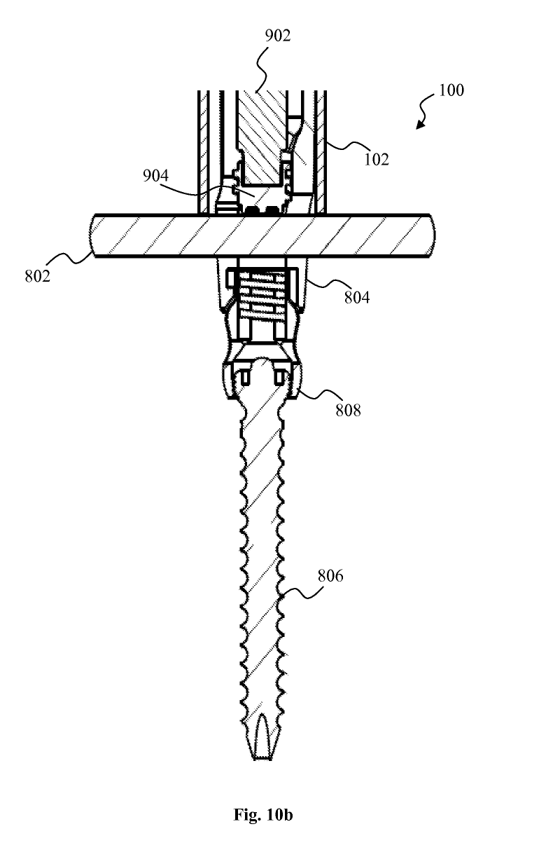

With additional reference to FIGS. 10a and 10b, an enlarged upper portion and an enlarged lower portion, respectively, of the cross-sectional view of FIG. 9 are illustrated. As can be seen, FIG. 10 provides a view of the interaction between the sleeve 102, reduction handle 104, threaded boss member 106, drive transmission 108, extension 804, and driver 902.

The extension 804 may extend through the sleeve 102 and threadingly engage the inner threaded portion 412 of the threaded boss member 106. For example, the inner threaded portion 412 may engage threads on the interior of the extension 804. The sleeve 102, reduction handle 104, threaded boss member 106, and drive transmission 108 may be coupled as previously described. The driver 902 may extend from the proximal portion 402 of the threaded boss member 106 to the distal end 204 of the sleeve 102. A proximal portion of the driver 902 may fit within a bore in the distal end 504 of the drive transmission 108. A distal end of the driver 902 may engage the locking cap 904. As illustrated in FIG. 10b, the sleeve 102 may contact the rod 802

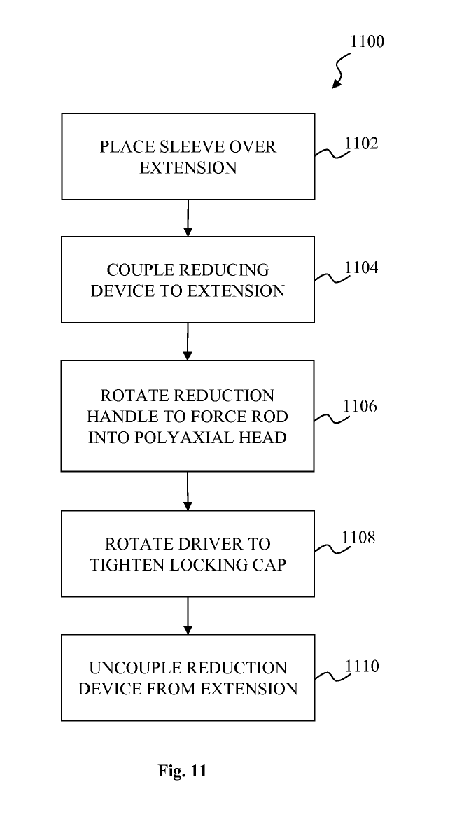

Referring to FIG. 11, a method 1100 illustrates one embodiment of a process using the reduction device 100 of FIG. 1. In step 1102, the sleeve 102 of the reduction device 100 may be placed over the extension 804. In step 1104, the threaded boss member 106 may be rotated so that the inner threaded portion 412 engages the threads of the extension 804. As the extension 804 is coupled to the implanted bone anchor 806 via the polyaxial head 808, the extension may be unable to rotate relative to the bone anchor. As the threaded boss member 106 is coupled to the extension 804, the threaded boss member may be unable to rotate relative to the extension once tightened.

Accordingly, when the reduction handle 104 is turned in step 1106, the threaded boss member 106 that is threadingly engaged to the reduction handle may move upward relative to the reduction handle (e.g., towards the proximal end 202 of the sleeve 102). This movement in turn forces the rod 802, which is unable to enter the sleeve 102 in its illustrated orientation of FIG. 11, into the polyaxial head 808. In step 1108, once the rod 802 is firmly seated in the polyaxial head 808, the drive transmission 108 may then be rotated to transfer force via the driver 902 to the locking cap 904 and secure the locking cap to the polyaxial head 808. The reduction device 100 may be removed prior to final tightening of the locking cap 904 (e.g., using a force of approximately 110 inch pounds) or the final tightening may be performed with all or a portion of the reduction device still in place, as illustrated in step 1110.

In the present example, although the driver 902 engages the drive transmission 108, it may not be coupled to any portion of the reduction instrument 100. Accordingly, the reduction instrument 100 may be removed without removing the driver 902 from the locking cap 904. For example, the threaded boss member 106 may be unscrewed from the extension 804 and removed. As the drive transmission 108 is coupled to the threaded boss member 106 (e.g., via the tabs 508a-508d) and is not coupled to the driver 902, the drive transmission may be removed with the threaded boss member. After the threaded boss member 106 is uncoupled from the extension 804, the reduction handle 104 and sleeve 102 (which may be coupled via the tabs 208a-208d) are no longer attached to the extension and may be removed. Accordingly, during and after removal of the reduction instrument 100, the extension 804 and driver 902 may remain in position.

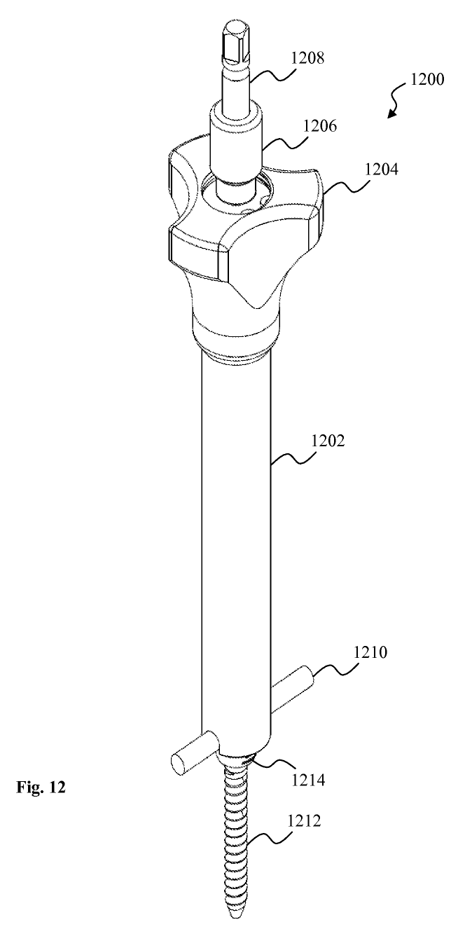

Referring to FIG. 12, in another embodiment, a reduction device 1200 is illustrated. The reduction device 1200 may include a sleeve 1202, a reduction handle 1204, and a threaded boss member 1206. As with the reduction device 100 of FIG. 1, a driver 1208 may extend through the reduction device 1200 to apply pressure to seat a rod 1210 into a screw head 1214 that is coupled to a screw 1212 during a spinal surgery procedure.

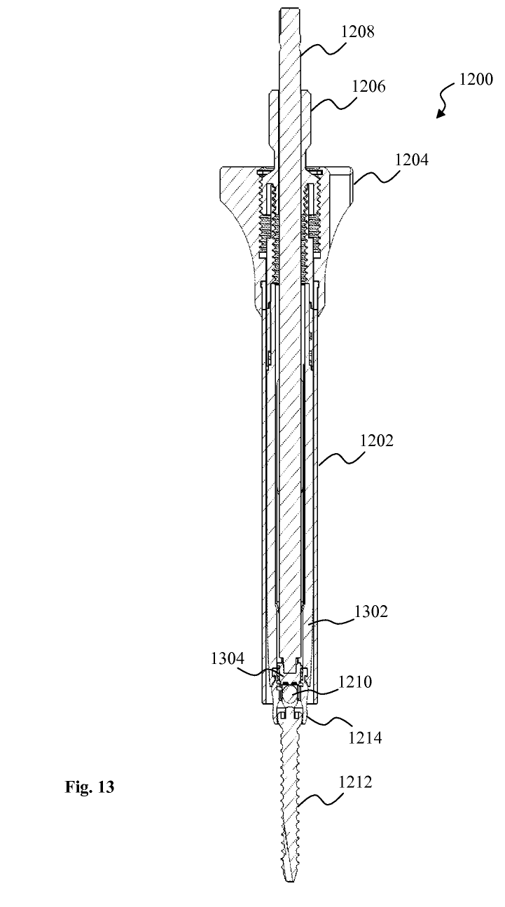

Referring to FIG. 13, a cross-sectional view of one embodiment of the reduction device 1200 of FIG. 12 is illustrated. As can be seen, the reduction device 1200 may accept an extension 1302. A locking cap 1304 is also illustrated. As the extension 1302 may be similar or identical to the extension 804 of FIG. 8 and the locking cap 1304 may be similar or identical to the locking cap 904 of FIG. 9, they will not be further described.

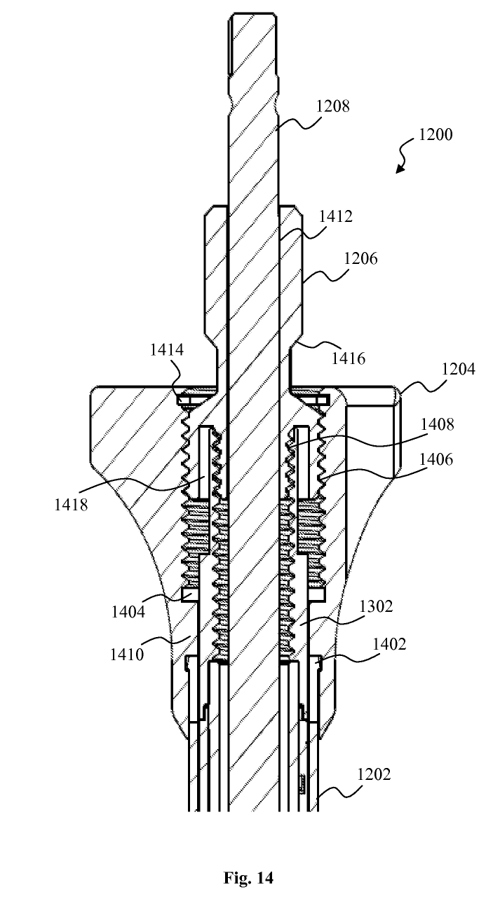

Referring to FIG. 14, an enlarged upper portion of the cross-sectional view of FIG. 13 is illustrated. As can be seen, FIG. 14 provides a view of the interaction between the sleeve 1202, reduction handle 1204, threaded boss member 1206, driver 1208, and extension 1302.

The sleeve 1202 may be similar to the sleeve 102 of FIG. 1. For example, the sleeve 1202 may include a lip 1402 on a proximal end of the sleeve to engage a groove in the reduction handle 1204. This enables the sleeve 1202 to rotate with respect to the reduction handle 1204. The sleeve 1202 may also include other features previously discussed, such as one or more tabs (not shown).

The reduction handle 1204 may include a partially or completely threaded bore 1404 that passes through the reduction handle. In the present example, an upper portion of the threaded bore 1404 is sized to accept an outer thread portion 1406 of the threaded boss member 1206. A lower portion of the threaded bore 1404 may have a larger diameter than the upper portion, and may be smooth with a slot or other indentation to accept the lip 1402 of the sleeve 1202.

The sleeve 1202 may fit into the bore 1404 of the reduction handle 1204. When inserted, the lip 1402 may fit into the groove in the lower smooth portion of the bore 1404. In addition, the bore 1404 may be slightly narrower above the groove. For example, a stop 1410 may be formed within the bore 1404. The stop 1410 may limit the downward motion of the threaded boss member 1206 and/or the upward movement of the extension 1302. Accordingly, the sleeve 1202 may freely rotate with respect to the reduction handle 1204 while still retaining the reduction handle.

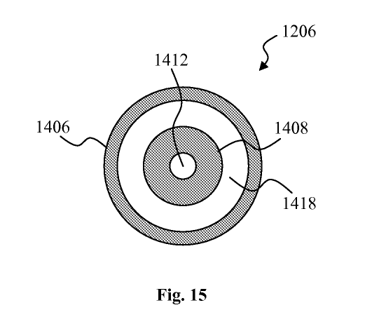

With additional reference to FIG. 15, which illustrates a cross-section of the threaded boss member 1206 from the top of the reduction device 1200, the threaded boss member may include an inner threaded portion 1408 and an outer threaded portion 1406. The inner threaded portion 1408 may engage the extension 1302, as was described with respect to the threaded boss member 106 of FIG. 1. The outer threaded portion 1406 may engage the threaded bore 1404 of the reduction handle 1204. In the present embodiment, the inner threaded portion 1408 and the outer threaded portion 1406 may form two concentric circles that are open towards one end (e.g., the end oriented towards the distal end of the sleeve 1202) and that intersect a substantially planar surface at the other end. Accordingly, the threaded boss member 1206 may form a “cap” that includes an open end with the threaded concentric circles. A gap 1418 may exist between the two concentric circles to allow the extension 1302 to move between the circles and engage the inner threaded portion 1408. A bore 1412, which may be smooth as illustrated, may extend through the threaded boss member 1206 to allow for passage of the driver 1208.

Referring again specifically to FIG. 14, in the present example, a collar 1414 may be provided to limit the downward movement of the threaded boss member 1206. For example, the collar 1414 may engage a shoulder 1416 on the threaded boss member 1206 to prevent downward movement once the two surfaces make contact.

In operation, the sleeve 1202 of the reduction device 1200 may be placed over the extension 1302. The threaded boss member 1206 may be rotated so that the inner threaded portion 1408 engages the threads of the extension 1302. As the extension 1302 is coupled to the implanted bone anchor 1212, the extension may be unable to rotate relative to the bone anchor. As the threaded boss member 1206 is coupled to the extension 1302, the threaded boss member may be unable to rotate relative to the extension. Accordingly, when the reduction handle 1204 is turned, the threaded boss member 1206 that is threadingly engaged to the reduction handle may move upward relative to the reduction handle. This movement in turn forces the rod 1210, which is unable to enter the sleeve 1202 in the orientation illustrated in FIG. 12, into the polyaxial head 1214. Once the rod 1210 is firmly seated in the polyaxial head 1214, the driver 1208 may then be rotated to transfer force to the locking cap 1304 and secure the locking cap to the polyaxial head 1214.

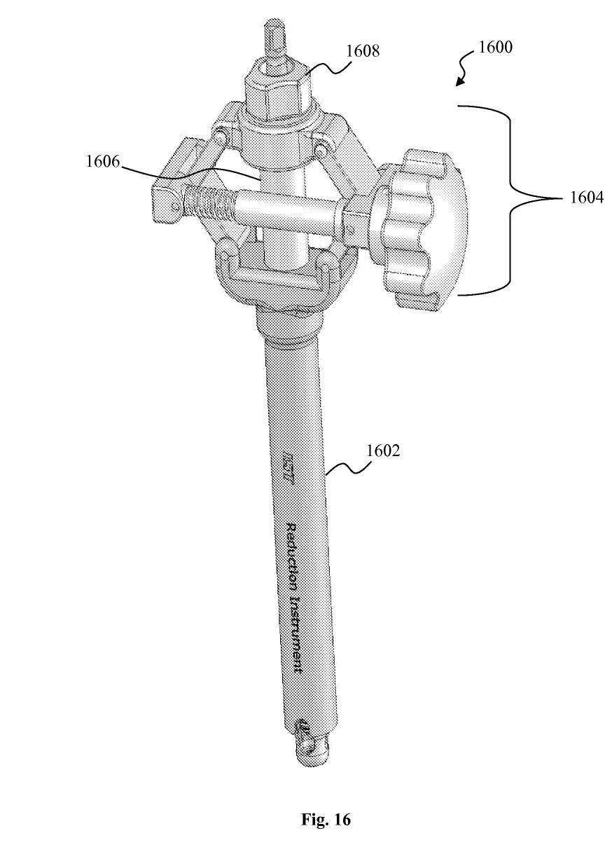

Referring to FIG. 16, in another embodiment, a reduction device 1600 is illustrated. The reduction device 1600 may include a sleeve 1602, a linkage assembly 1604, a coupling member 1606, and gripping surface 1608. As will be discussed below in greater detail, the reduction device 1600 may be used to apply pressure to seat a rod in a screw head during a spinal surgery procedure. A locking cap may then be tightened, either with all or a portion of the reduction device 1600 in place or after the reduction device has been removed, to secure the rod to the screw head. Accordingly, the sleeve 1602, linkage assembly 1604, coupling member 1606, and gripping surface (e.g., a knob) 1608 may interact to provide the force needed to seat the rod while eliminating or minimizing stress to the threads of the locking cap. FIG. 16 also illustrates a polyaxial head, which will be described in greater detail below.

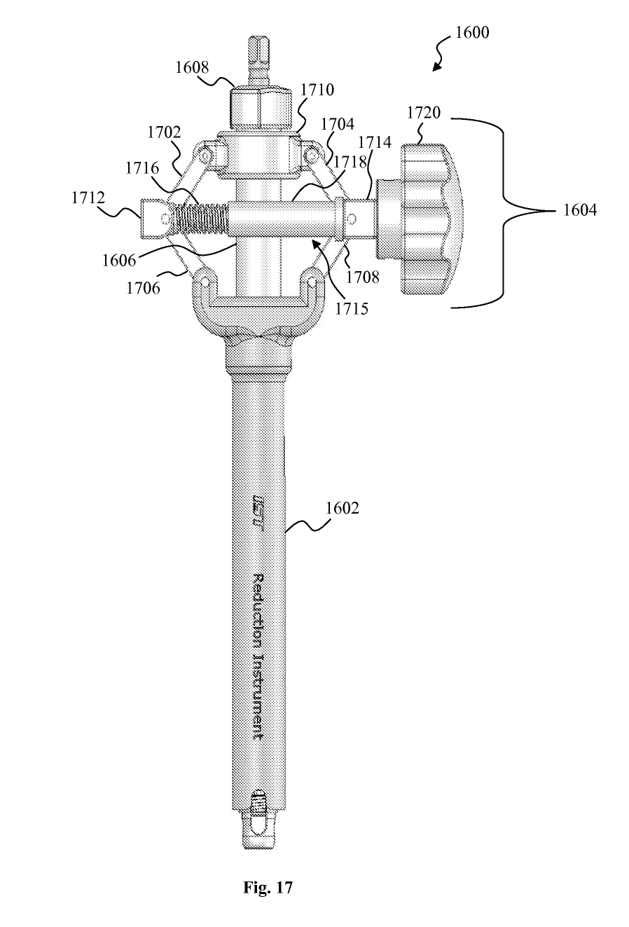

Referring to FIG. 17, a side view of one embodiment of the reduction device 1600 of FIG. 16 is illustrated. In the present example, the linkage assembly 1604 may include links 1702, 1704, 1706, and 1708. The links 1702 and 1704 may be pivotally coupled to a collar 1710 and the links 1706 and 1708 may be pivotally coupled to the sleeve 1702. The links 1702 and 1706 may also be pivotally coupled to a cross member 1712 and the links 1704 and 1708 may be pivotally coupled to a cross member 1714. A threaded member 1715 may couple the cross members 1712 and 1714. In the present example, the threaded member 1715 may include a member 1716 having exterior threads that may be pivotally coupled to the cross member 1712, and a member 1718 having a threaded opening that may be pivotally coupled to the cross member 1714. A gripping surface 1720 (e.g., a knob) may be coupled to the member 1718.

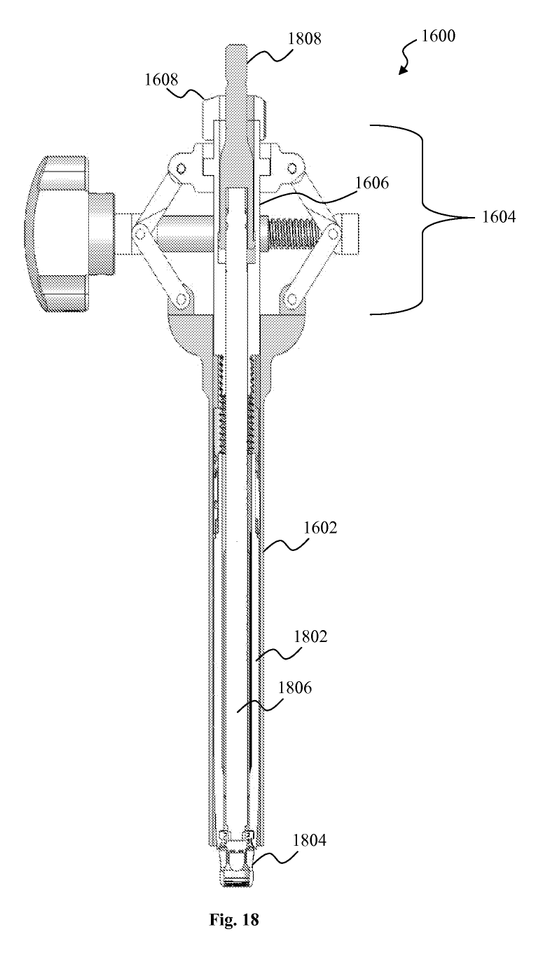

With additional reference to FIG. 18, a cross-sectional view of the reduction device of FIG. 17 is illustrated from the opposite side. The coupling member 1606 may threadingly engage an extension 1802 that may be coupled to a polyaxial head 1804. A tool 1806 may extend down the extension 1802. A driver 1808 may be coupled to the tool 1806.

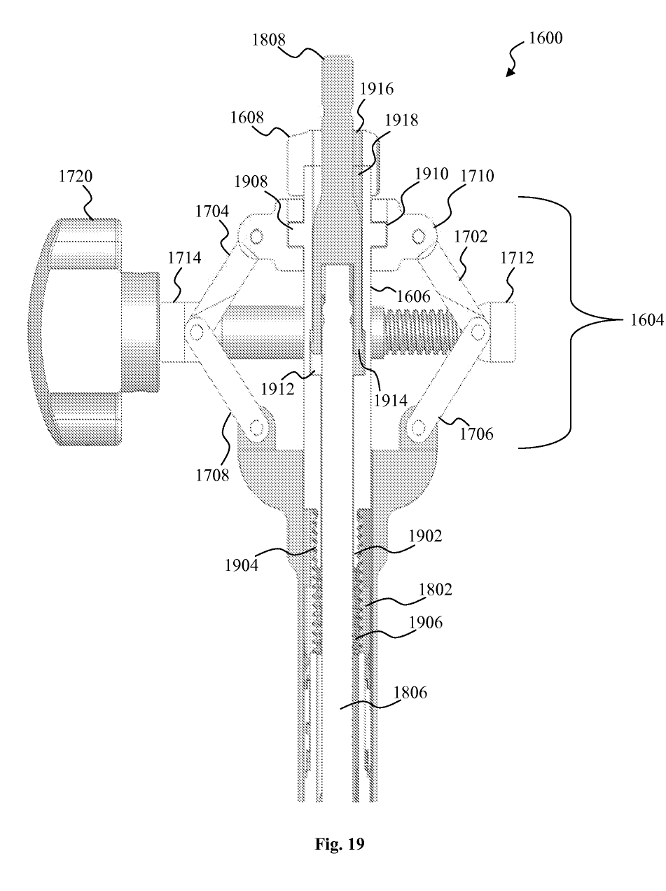

With additional reference to FIG. 19, a close up view of an upper portion of the reduction device of FIG. 18 is illustrated. The coupling member 1606 may include a lower portion 1902 having external threads 1904. The threads 1904 may engage threads 1906 of the extension 1802. In the present example, the lower portion 1902 may have a smaller diameter than the upper portion of the coupling member 1606. The coupling member 1606 may be coupled to the linkage assembly 1604 at the collar 1710. In the present example, the coupling member 1606 includes a flange 1908 that extends into an internal groove 1910 of the collar 1710. The flange 1908 may freely rotate within the groove 1910, enabling the coupling member 1606 to freely rotate with respect to the linkage assembly 1604. The coupling member 1606 may be securely joined to the knob 1608. Accordingly, rotation of the knob 1608 may result in rotation of the coupling member 1606. In this manner, the coupling member 1606 may be threaded into the extension 1802 without the need to rotate the linkage assembly 1604.

The coupling member 1606 may have an interior groove 1912. The groove 1912 may receive a flange 1914 of the driver 1808. The flange 1914 may snap into the groove 1912 when the driver is inserted into a bore 1916 of the knob and a bore 1918 of the coupling member 1606. Accordingly, the flange 1914 and the groove 1912 may retain the driver 1808 in the coupling member 1606. In the present example, the driver 1808 may rotate with respect to the coupling member 1606.

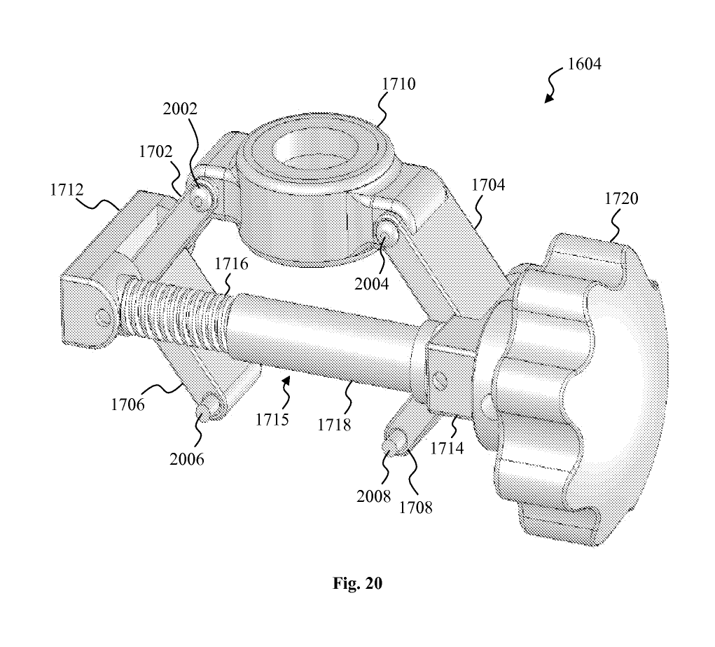

Referring to FIG. 20, a perspective view of one embodiment of the linkage assembly 1604 (FIG. 16) is illustrated. In the present example, the links 1702, 1704, 1706, and 1708 may be solid, but it is understood that the links may not be solid in other embodiments. For example, each link 1702, 1704, 1706, and 1708 may be two or more links or may be formed from multiple support members. The links 1702 and 1704 may be coupled to the collar 1710 by pins 2002 and 2004, respectively. The links 1706 and 1708 may be coupled to the sleeve 1602 (FIG. 16) by pins 2006 and 2008, respectively. It is understood that each pin 2002, 2004, 2006, and 2008 may be multiple pins, and so a separate pin may couple each link to the collar 1710 and/or the sleeve 1602.

The actual configuration of the linkage assembly 1604 may vary. For example, the links 1702, 1704, 1706, and 1708 and/or the member 1716 may be lengthened or shortened. Furthermore, the thread form of the member 1716 may be varied to increase or decrease the number of rotations needed to move the linkage assembly 1604 from a fully closed position to a fully open position. In still other embodiments, a member (not shown) capable of telescoping or otherwise lengthening/shortening may replace the member 1716 and the member 1716 may be configured to adjust the telescoping member. In yet other embodiments, the telescoping member may be adjusted without use of the member 1716. Accordingly, different mechanisms may be employed to manipulate the links 1702, 1704, 1706, 1708.

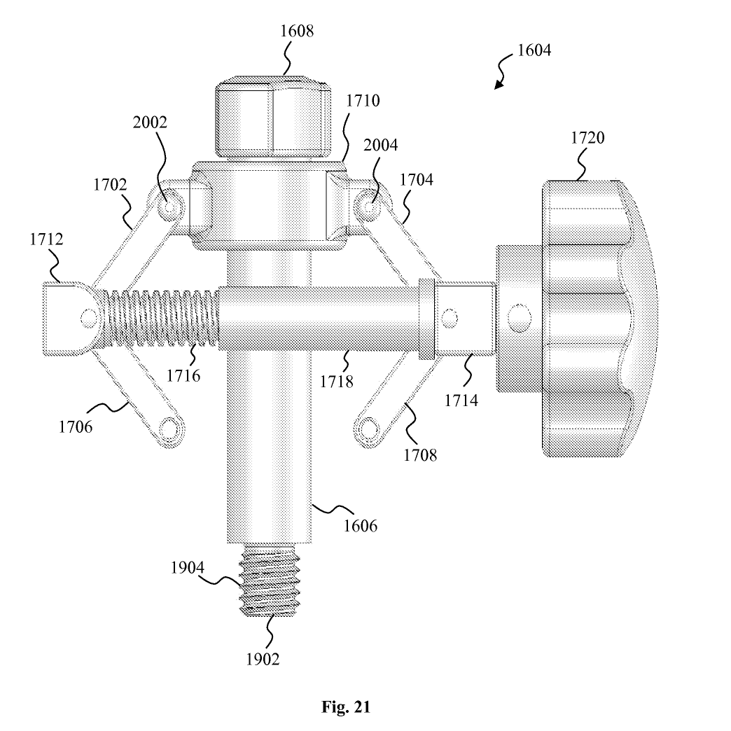

Referring to FIG. 21, a side view of one embodiment of the linkage assembly 1604, coupling member 1606, and knob 1608 of FIG. 6 is illustrated. The lower portion 1902 and the threads 1904 of the lower portion may threadingly engage the extension 1802 (FIG. 18) as previously described. The links 1706 and 1708 may pivotally engage the sleeve 1602.

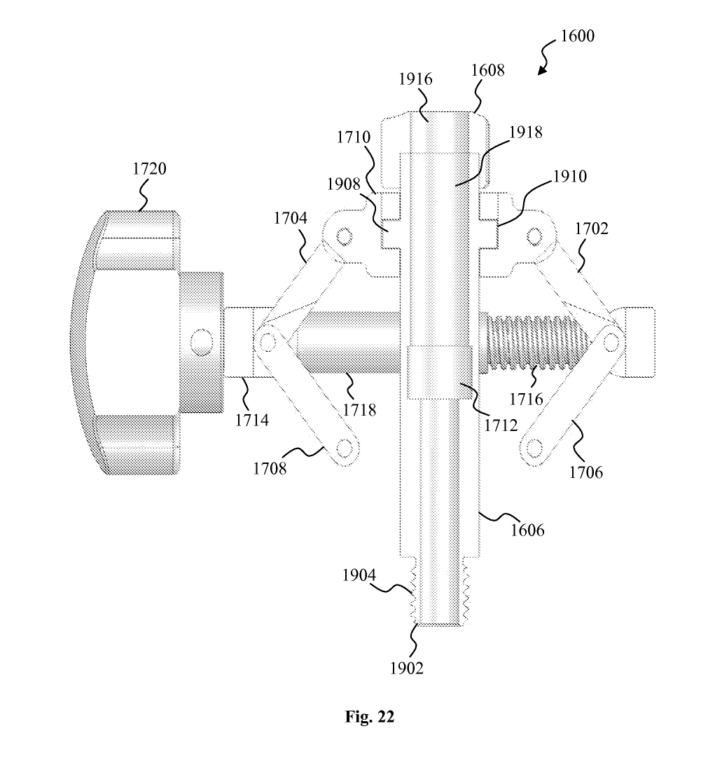

With additional reference to FIG. 22, a cross-sectional view of FIG. 21 is illustrated from the opposite side. As described previously, the bore 1918 may include a groove 1912 for receiving the flange 1914 (FIG. 19) of the driver 1808. This enables the coupling member 1606 to rotate independently relative to the linkage assembly 1604.

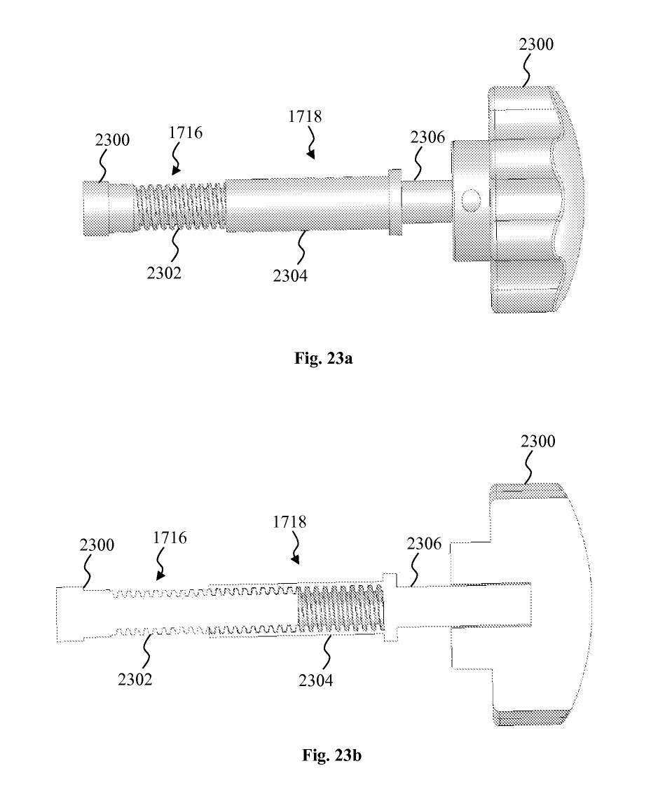

Referring to FIGS. 23a and 23b, a side view and a cross-sectional view, respectively, of a portion of the linkage assembly 1604 are illustrated. In the present example, the member 1716 may include a neck 2300 and a threaded portion 2302. The neck 2300 may be coupled to the cross member 1712. In the present embodiment, the neck 2300 may rotate with respect to the cross member 1712, or the neck may rotate with respect to the threaded portion 2302. The member 1718 may include a threaded portion 2304 having an internally threaded opening for mating with the threaded portion 2302. The member 1718 may include a neck 2306 that may couple to the cross-member 1714. The neck 2306 may rotate with respect to the cross member 1714, or the neck may rotate with respect to the threaded portion 2304.

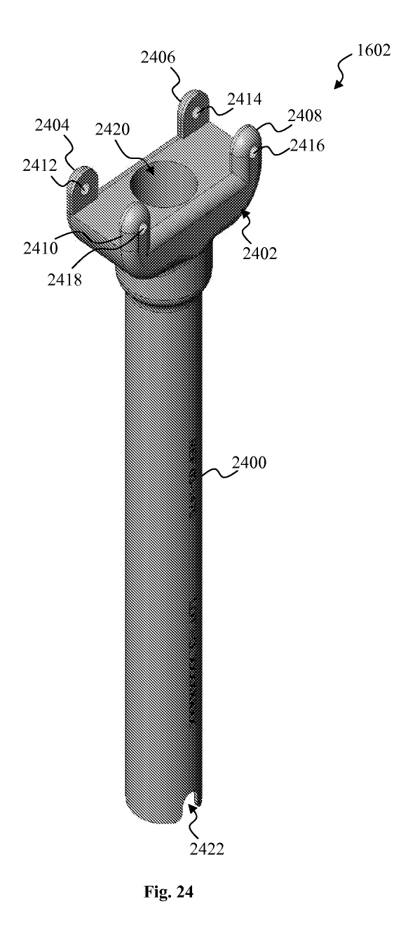

Referring to FIG. 24, one embodiment of the sleeve 1602 of FIG. 16 is illustrated. The sleeve 1602 may include a shell 2400 coupled to a connector portion 2402. The connector portion 2402 may include multiple protrusions 2404, 2406, 2408, and 2410 having openings 2412, 2414, 2416, and 2418, respectively. The openings 2412 and 2418 may receive the pin 2006 (FIG. 20) and the openings 2414 and 2416 may receive the pin 2008 (FIG. 20). A bore 2420 may extend through the connector portion 2402 and shell 2400. In the present example, an indentation 2422 in the distal portion of the shell 2400 and a matching indentation (not shown) opposite the indentation 2422 may receive a rod during a surgical procedure, as will be described below.

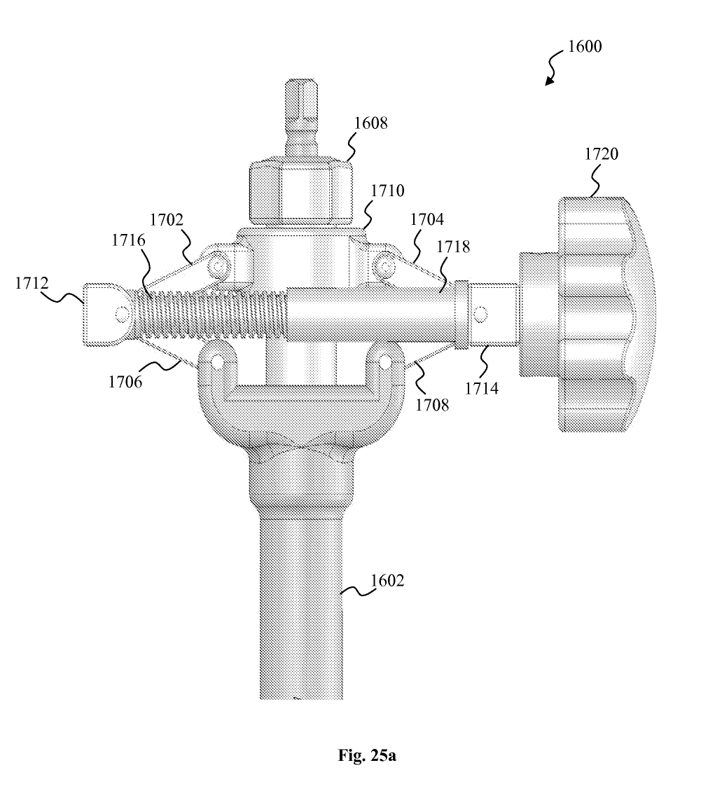

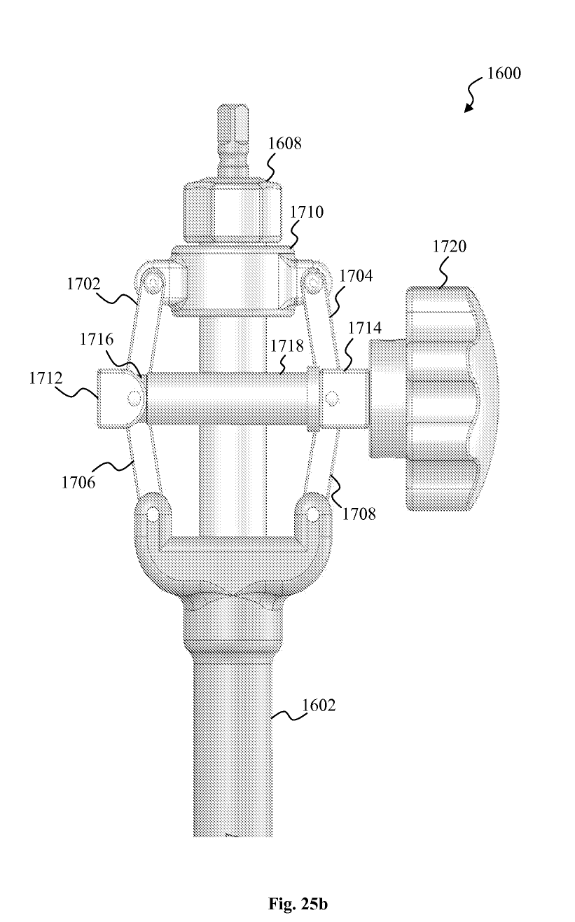

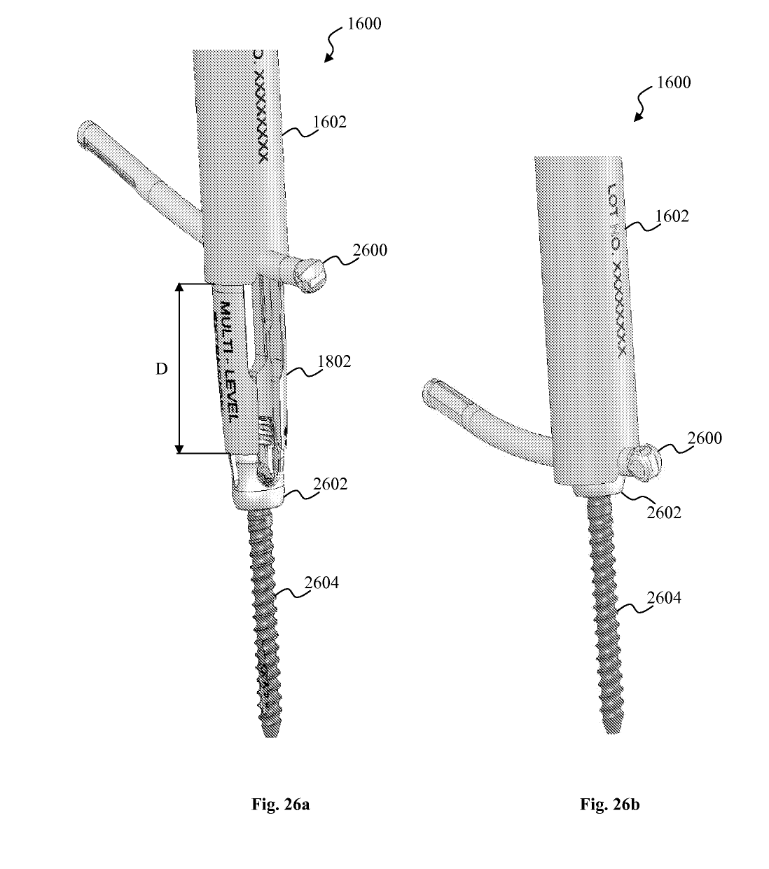

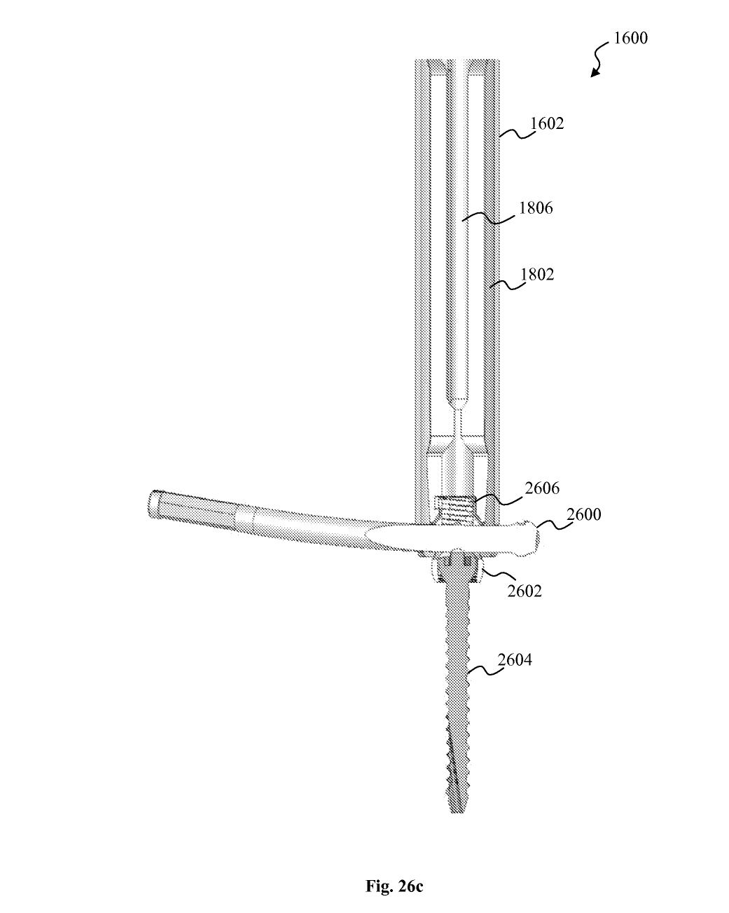

Referring to FIGS. 25a and 26a, an embodiment of an upper portion and a lower portion, respectively, of the reduction instrument of FIG. 16 is illustrated. In the present example, as illustrated in FIG. 25a, the linkage assembly 1604 has pulled the sleeve 1602 into an upper position (e.g., upward and toward the collar 1710). As illustrated in FIG. 26a, this may result in a distal end of the sleeve 1602 being a maximum distance D from the bottom of the extension 1802 that is coupled to a bone anchor 2604 via a polyaxial head 2602. It is understood that the sleeve 1602 may not be moved to the maximum distance D in some embodiments. The indentations 2422 (FIG. 24) of the sleeve 1602 may receive a rod 2600. Examples of the rod 2600, extension 1802, and polyaxial head 2602 are described in previously incorporated U.S. patent application Ser. Nos. 10/690,211; 10/990,272; and 10/989,715.

Referring to FIGS. 25b and 26b, an embodiment of an upper portion and a lower portion, respectively, of the reduction instrument of FIG. 16 is illustrated. In the present example, as illustrated in FIG. 25b, the linkage assembly 1604 has pushed the sleeve 1602 into a lower position (e.g., downward and away from the collar 1710). As illustrated in FIG. 26b, this may result in a distal end of the sleeve 1602 being a minimum distance from the polyaxial head 2602, thereby forcing the rod 2600 into the polyaxial head.

Referring to FIG. 26c, a cross-sectional view of the reduction device of FIG. 26b is illustrated with the rod 2600, polyaxial head 2602, and bone anchor 2604. Once the rod 2600 is in place (as illustrated in FIGS. 26b and 26c), the tool 1806 may be rotated (e.g., via the driver 1608) to secure a locking cap 2606 to the polyaxial head 2602, locking the rod into place.

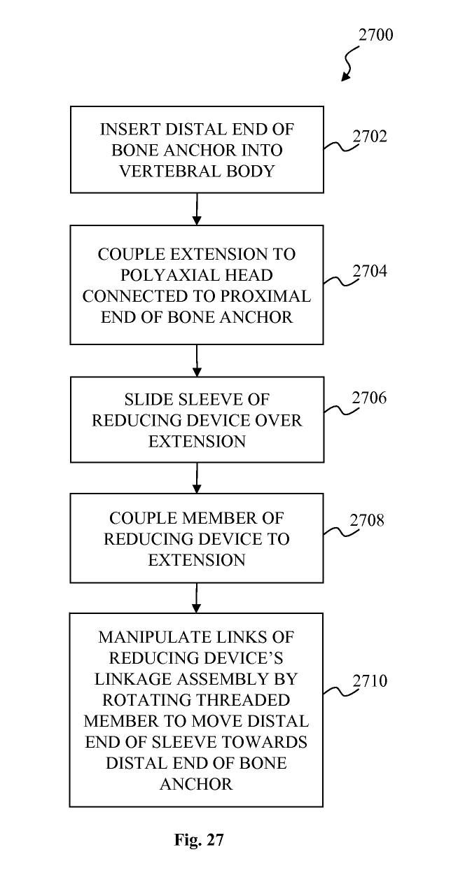

Referring to FIG. 27, a method 2700 illustrates one embodiment of a surgical procedure that may use the reduction device of FIG. 16. In step 2702, a distal end of a bone anchor (e.g., a pedicle screw such as the screw 2604 of FIGS. 26a-26c) may be inserted into a vertebral body. In step 2704, the extension 1802 (FIG. 18) may be coupled to a polyaxial head (e.g., the polyaxial head 1602 of FIGS. 26a-26c) connected to a proximal end of the bone anchor. In step 2706, the sleeve 1602 of the reduction device 1600 may be placed over the extension 1802. In step 2708, the coupling member 1606 of the reducing device 1600 may be coupled to the extension 1802 within the sleeve 1602. As described previously, the coupling member 1606 may be coupled to the sleeve 1602 via the linkage assembly 1604. In step 2710, the linkage assembly 1604 may be manipulated to move a distal end of first and second links of the linkage assembly away from a distal end of third and fourth links of the linkage assembly. For example, the manipulation may be accomplished by rotating a threaded member 1715 of the linkage assembly 1604 coupled to the first, second, third, and fourth links. The moving may force a distal end of the sleeve 1602 towards the distal end of the bone anchor 2604. The distal end of the sleeve 1602 may initially be raised to expose a portion of the extension 1802 (FIG. 26a) and may cover all or a portion of the polyaxial head 2602 when the rod 2600 is in place.

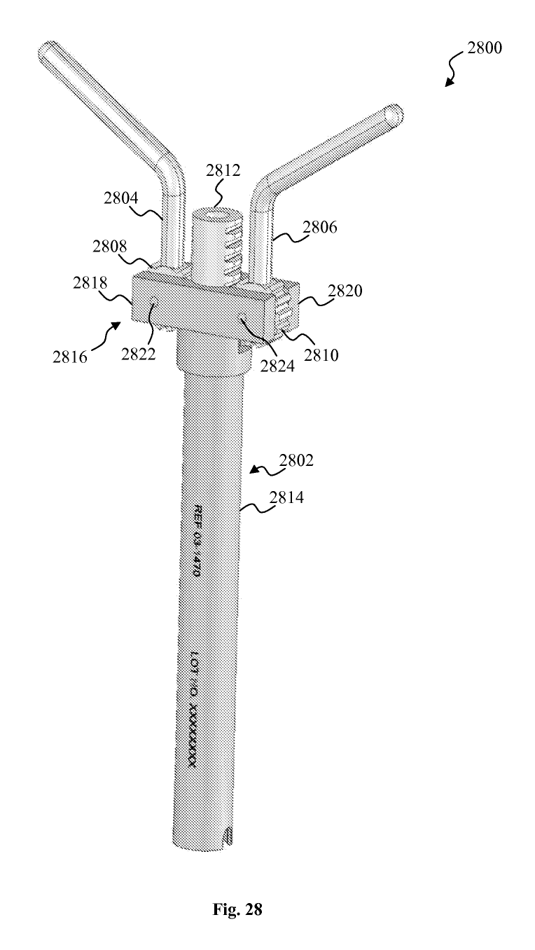

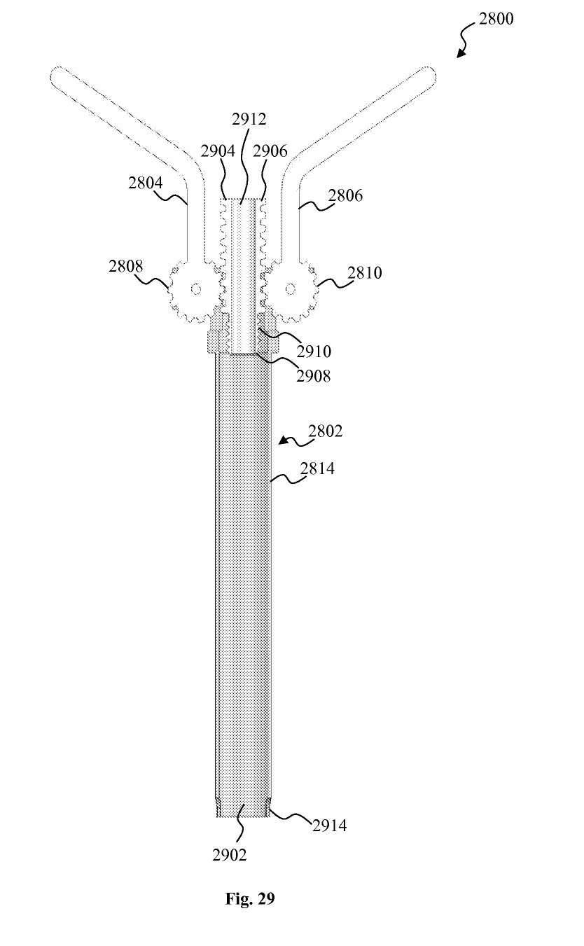

Referring to FIGS. 28 and 29, another embodiment of a reduction device 2800 is illustrated in a perspective view and a cross-sectional view, respectively. The reduction device 2800 may include a sleeve 2802, arms 2804 and 2806 coupled to gears 2808 and 2810, respectively, and a coupling member 2812. As will be discussed below in greater detail, the reduction device 2800 may be used to apply pressure to seat a rod in a screw head during a spinal surgery procedure. A locking cap may then be tightened, either with all or a portion of the reduction device 2800 in place or after the reduction device has been removed, to secure the rod to the screw head. Accordingly, the sleeve 2802, arms 2804 and 2806, gears 2808 and 2810, and coupling member 2812 may interact to provide the force needed to seat the rod while eliminating or minimizing stress to the threads of the locking cap.

The sleeve 2802 may include shell 2814 and a connector portion 2816. The connector portion 2816 may include cross members 2818 and 2820 that may be pivotally coupled to gears 2808 and 2810 by pins 2822 and 2824, respectively. A bore 2902 (FIG. 29) may extend through the shell 2802 and connector portion 2816. Indentations 2914 may be provided in a distal portion of the shell 2814 to receive a rod (not shown).

The coupling member 2812 may include teeth 2904 and 2906 facing the gears 2808 and 2810, respectively. The coupling member 2812 may include a lower portion 2908 having threads 2910 for threadingly engaging an extension (not shown).

In operation, the arms 2804 and 2806 may be moved upward/downward, thereby rotating gears 2808 and 2810, respectively. The gears 2808 and 2810 may engage the teeth 2904 and 2906, respectively, to move the sleeve 2802 relative to the coupling member 2812.

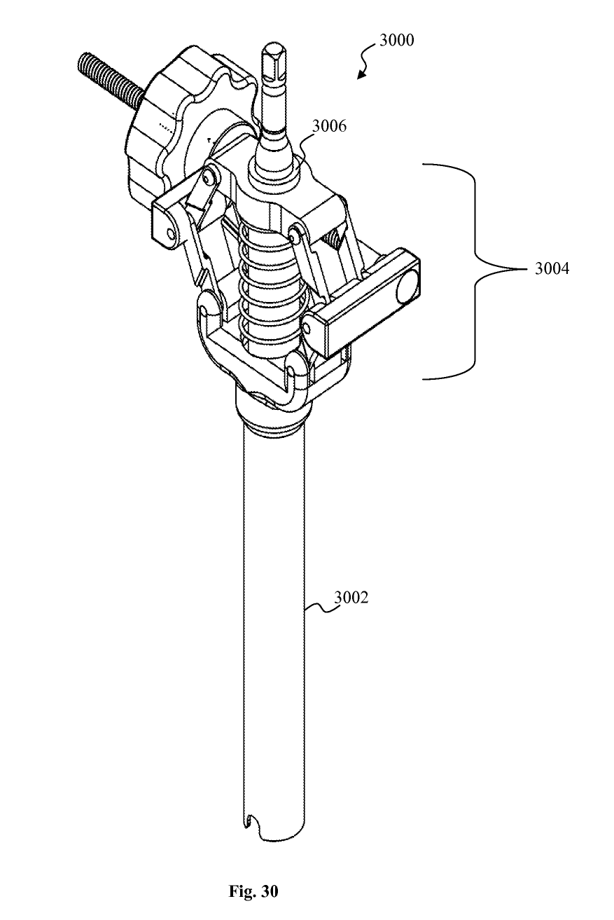

Referring to FIG. 30, in another embodiment, a reduction device 3000 is illustrated. The reduction device 3000 may include a sleeve 3002, a linkage assembly 3004, and a coupling member 3006. As will be discussed below in greater detail, the reduction device 3000 may be used to apply pressure to seat a rod in a screw head during a spinal surgery procedure. A locking cap may then be tightened, either with all or a portion of the reduction device 3000 in place or after the reduction device has been removed, to secure the rod to the screw head. Accordingly, the sleeve 3002, linkage assembly 3004, and coupling member 3006 may interact to provide the force needed to seat the rod while eliminating or minimizing stress to the threads of the locking cap.

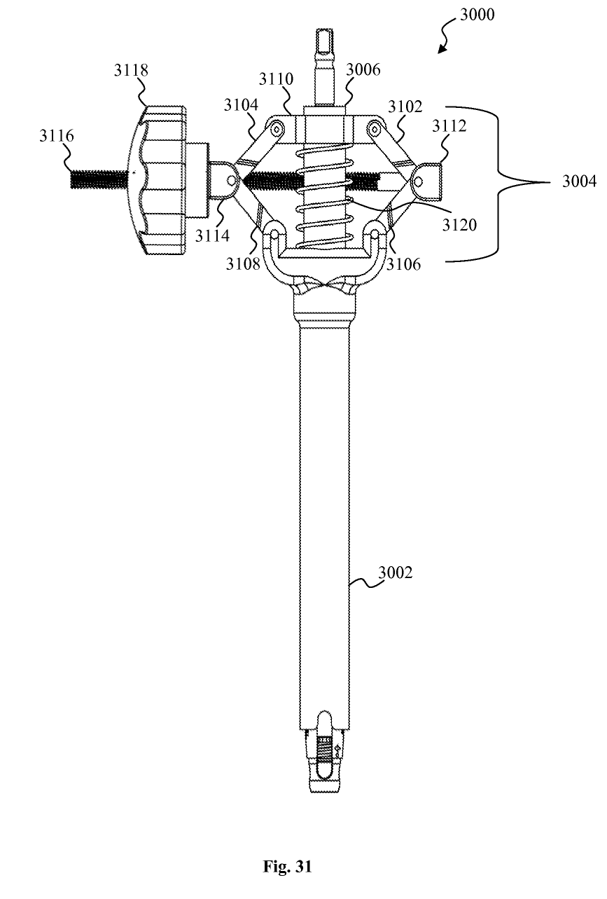

Referring to FIG. 31, a side view of one embodiment of the reduction device 3000 of FIG. 30 is illustrated. In the present example, the linkage assembly 3004 may include links 3102, 3104, 3106, and 3108. The links 3102 and 3104 may be pivotally coupled to a collar 3110 and the links 3106 and 3108 may be pivotally coupled to the sleeve 3002. The links 3102 and 3106 may also be pivotally coupled to a cross member 3112 and the links 3104 and 3108 may be pivotally coupled to a cross member 3114. A threaded member 3116 having exterior threads may be pivotally coupled to the cross members 3112 and 3114, and a gripping surface 3118 (e.g., a knob) may be coupled to the member 3116. In the present example, the knob 3118 may include a threaded bore (not shown) configured to threadingly engage the member 3116. The knob 3118 may also be coupled to the cross member 3114, and may rotate with respect to the cross member 3114. In some embodiments, a spring 3120 may encircle at least a portion of the coupling member 3006.

With additional reference to FIG. 32, a cross-sectional view of the reduction device of FIG. 30 is illustrated. The coupling member 3006 may threadingly engage an extension 3202 that may be coupled to a polyaxial head 3204. A tool 3206 may extend down the extension 3202. A driver 3208 may be coupled to the tool 3206. The driver 3208 may rotate freely with respect to the coupling member 3006. The collar 3110 and an upper portion of the sleeve 3002 may include indentations for capturing the spring 3120.

In operation, the knob 3118 may be rotated and, due to the threaded bore formed therein, may move the member 3116 relative to the knob. The movement of the member 3116, as it is coupled to the cross members 3112 and 3114, may result in movement of the links 3102, 3104, 3106, and 3108. Such movement may result in positioning of the links 3102, 3104, 3106, and 3108, which are pivotally coupled to the ring 3110 and sleeve 3002, into a substantially vertical position, a substantially horizontal position, or a position therebetween. In turn, this may move the sleeve 3002 relative to the coupling member 3006. Such movement may result in forcing a rod (not shown) into a polyaxial head (not shown).

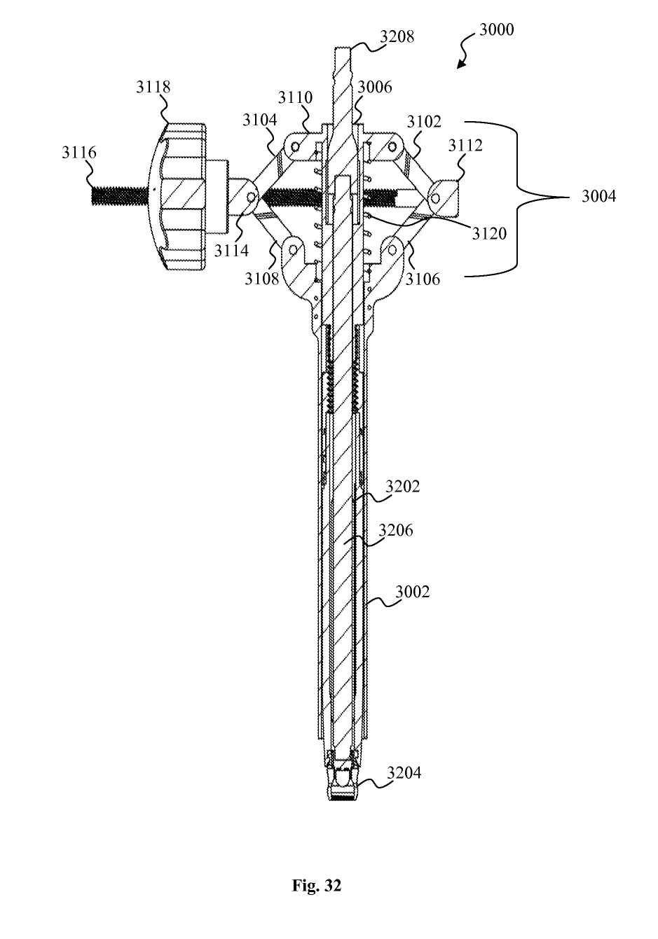

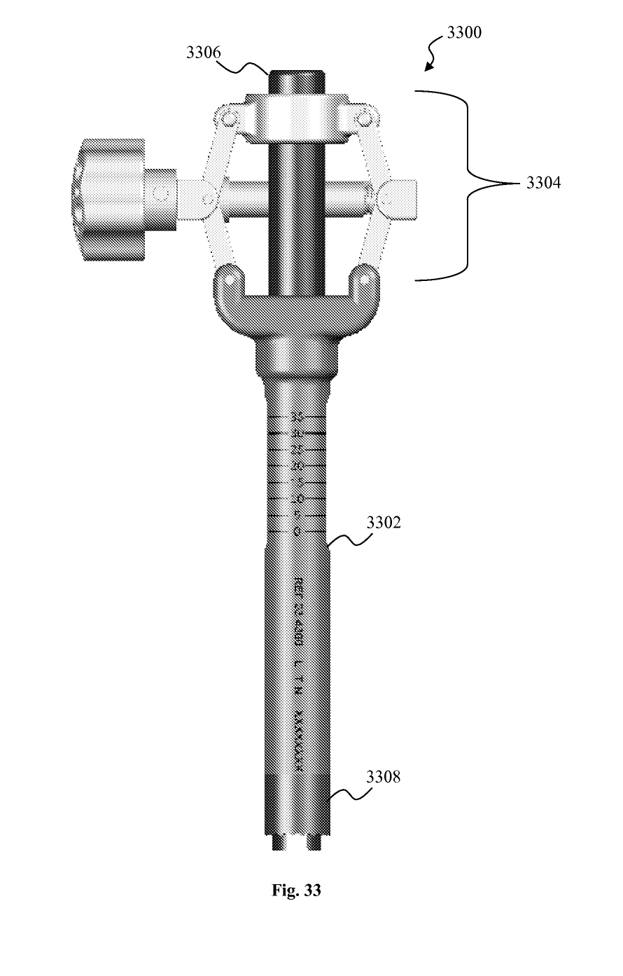

Referring to FIG. 33, in another embodiment, a reduction device 3300 is illustrated. The reduction device 3300 may include a sleeve 3302, a linkage assembly 3304, and a coupling member 3306. The reduction device 3300 may be used to apply pressure to seat a rod in a screw head during a spinal surgery procedure. A locking cap may then be tightened, either with all or a portion of the reduction device 3300 in place or after the reduction device has been removed, to secure the rod to the screw head. Accordingly, the sleeve 3302, linkage assembly 3304, and coupling member 3306 may interact to provide the force needed to seat the rod while eliminating or minimizing stress to the threads of the locking cap.

The linkage assembly 3304 may be similar or identical to the linkage assembly 1604 described previously with respect to the reduction device 1600 of FIG. 16. Accordingly, neither the linkage assembly nor its operation are discussed in detail in the present embodiment. An extension 3308 may be present in some examples. The extension 3308 may be immovably coupled to the sleeve 3302 or may be coupled to the sleeve in such a manner as to allow a defined range of motion relative to the sleeve.

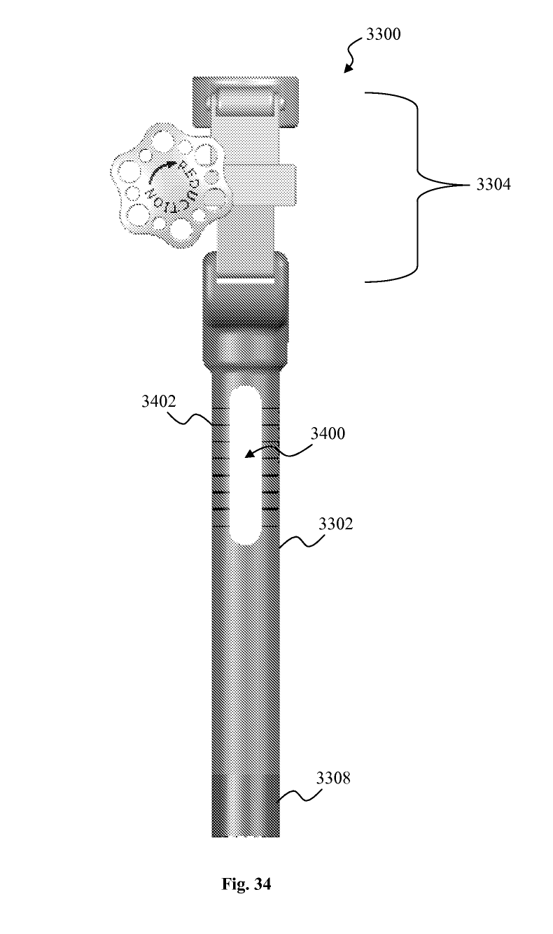

Referring to FIG. 34, a side view of one embodiment of the reduction device 3300 of FIG. 33 is illustrated. In the present example, the coupling member 3306 is absent to better illustrate a slot or other opening 3400 in the sleeve 3302. When present, the coupling member 3306 is visible through the opening 3400. An outer edge of the opening 3400 may include one or more markings 3402 to indicate, for example, positioning of the coupling member 3306 relative to the sleeve 3302.

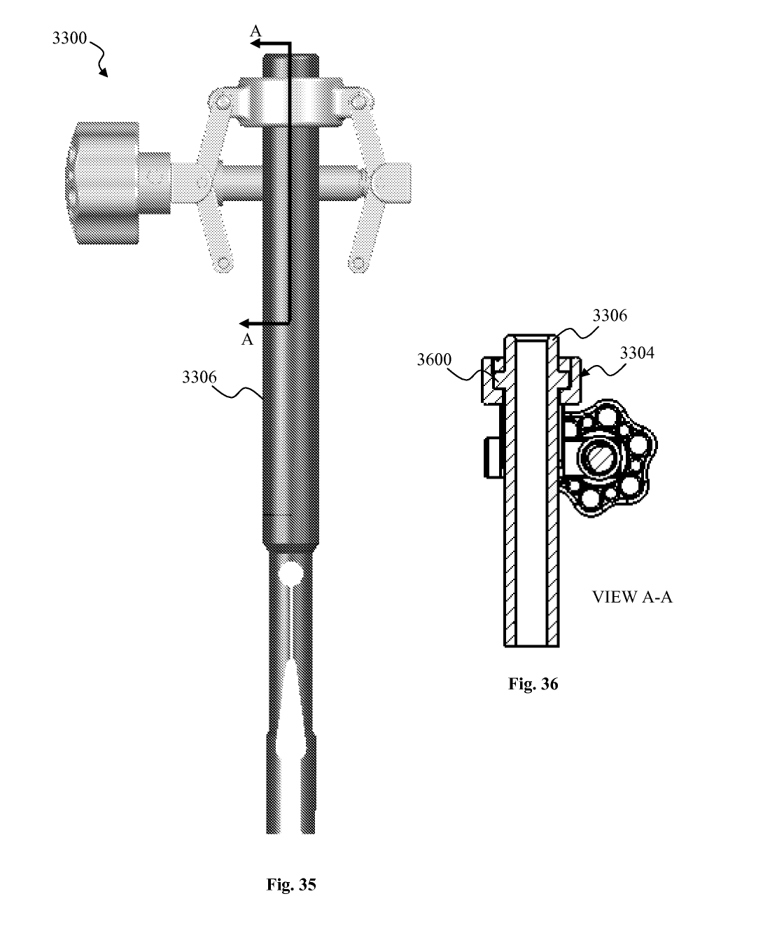

Referring to FIG. 35, a view of one embodiment of the reduction device 3300 of FIG. 33 is illustrated without the sleeve 3302 and extension 3308. In the present example, the coupling member 3306 may be a single component that is configured to couple to both the linkage assembly 3304 and to a polyaxial head or other bone anchor (not shown).

With additional reference to FIG. 36, a cross-sectional view along lines A-A of FIG. 35 is illustrated. Coupling member 3306 may include an upper ring or protrusion 3600 that engages the linkage assembly 3304 (e.g., the collar 1710 of FIG. 17). This enables the coupling member 3306 to rotate relative to the linkage assembly 3304 while maintaining a vertical relationship between the coupling member and the linkage assembly.

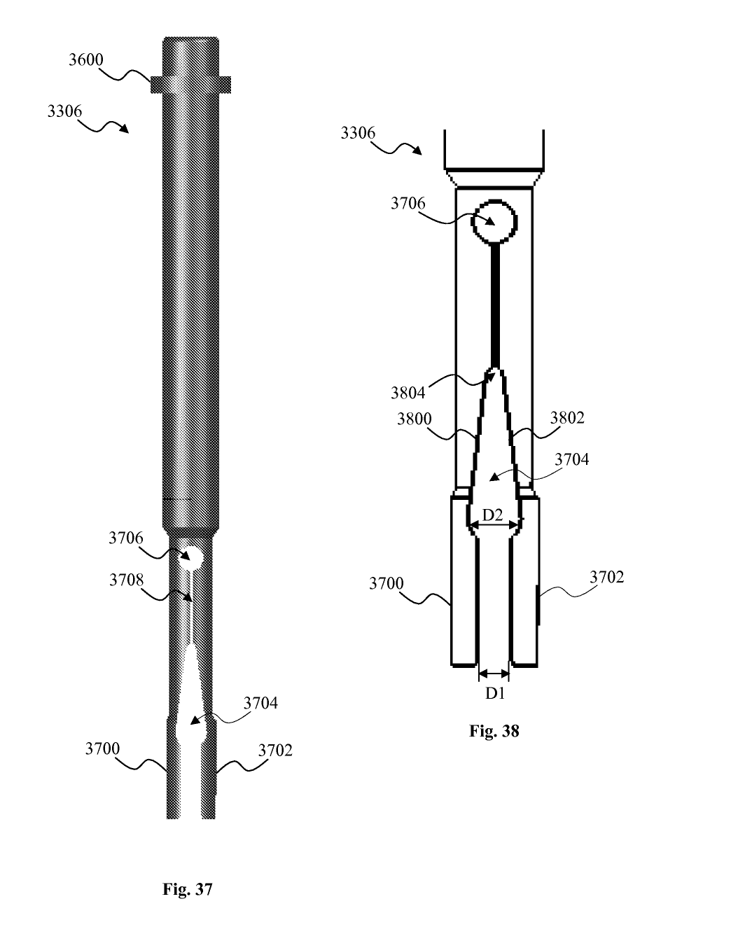

Referring to FIGS. 37 and 38, one embodiment of the coupling member 3306 is shown. In the present example, the distal end (relative to the linkage assembly 3304) of the coupling member 3306 includes two fingers 3700 and 3702. A space 3704 exists between the edges of the fingers 3700 and 3702. The space 3704 may be sized to allow a rod (e.g., the rod 2600 of FIGS. 26a-26c) to be moved into the coupling member 3306. Although not labeled in FIGS. 37 and 38, an identical space may be present between the edges of the fingers 3700 and 3702 on the opposite side of the coupling member 3306. An amount of movement between the fingers 3700 and 3702 may be controlled during design of the coupling member 3306 by altering the size of an opening 3706 that is connected to the space 3704 via slits 3708. For example, the opening 3706 may be designed to ensure clearance of the fingers 3700 and 3702 over a polyaxial head (not shown). Enlarging the opening 3706 may enable additional movement of the fingers 3700 and 3702 relative to one another. It understood that fingers 3700 and 3702 may be designed differently, and may be regulated in many different ways other than or in conjunction with the opening 3706.

With specific reference to FIG. 38, each finger 3700 and 3702 may include sloped or ramped sides 3800 and 3802, respectively, that are tapered with the proximal end of the opening 3704 being narrower than the distal end to form an inverted “V” shape when viewed from the perspective of FIG. 38. Accordingly, the narrower portion (indicated by reference number 3804) of the opening 3704 may be next to the slit 3708 and the opening may widen as it extends in the direction of the distal ends of the fingers 3700 and 3702. For purposes of illustration, the widest portion of the opening 3704 is labeled D2. Distal ends of the fingers 3700 and 3702 may be separated by a distance D1. In the present example, the distance D1 is defined to enable the fingers 3700 and 3702 to be coupled to a polyaxial head.

With additional reference to FIGS. 39 and 40, a perspective view and a cross-section view, respectively, of one embodiment of the distal end of the coupling member of FIG. 37 are illustrated. In the present example, at least a portion of the interior surfaces of the fingers 3700 and 3702 may be configured to provide protrusions 3900 and 3902, respectively. The protrusions 3900 and 3902 may be designed to enable the fingers 3700 and 3702 to couple to a polyaxial head or other bone anchor as will be described later in greater detail.

Referring specifically to FIG. 40, indentions 4000 and 4002 may be provided in fingers 3700 and 3702, respectively, to receive a portion of the polyaxial head. It is understood that the configuration of the inner surfaces of the fingers 3700 and 3702 may vary in order to attach to a particular polyaxial head or bone anchor. Accordingly, the illustrated distal end of the coupling member 3306 may have a variety of shapes and configurations to adapt to a variety of bone anchor systems.

Referring to FIG. 41, the distal end of the coupling member 3306 of FIG. 37 is illustrated with the extension 3308 of FIG. 33. In the present example, the extension 3308 may be coupled to the sleeve 3302 and may along a longitudinal axis of the coupling member 3306 when the sleeve is moved relative to the coupling member 3306 by actuation of the linkage assembly 3304.

Referring to FIG. 42, one embodiment of a ramp ring 4200 is illustrated that may be used with the coupling member 3306 of FIG. 37. In the present example, the ramp ring 4200 may fit over the outside of the fingers 3700 and 3702 and may slide up and down the fingers within a range D3. It is understood that the range D3 shown in the present example is for purposes of example and may be many different distances. The exterior surface of the ramp ring 4200 may abut the interior surface of the extension 3308.

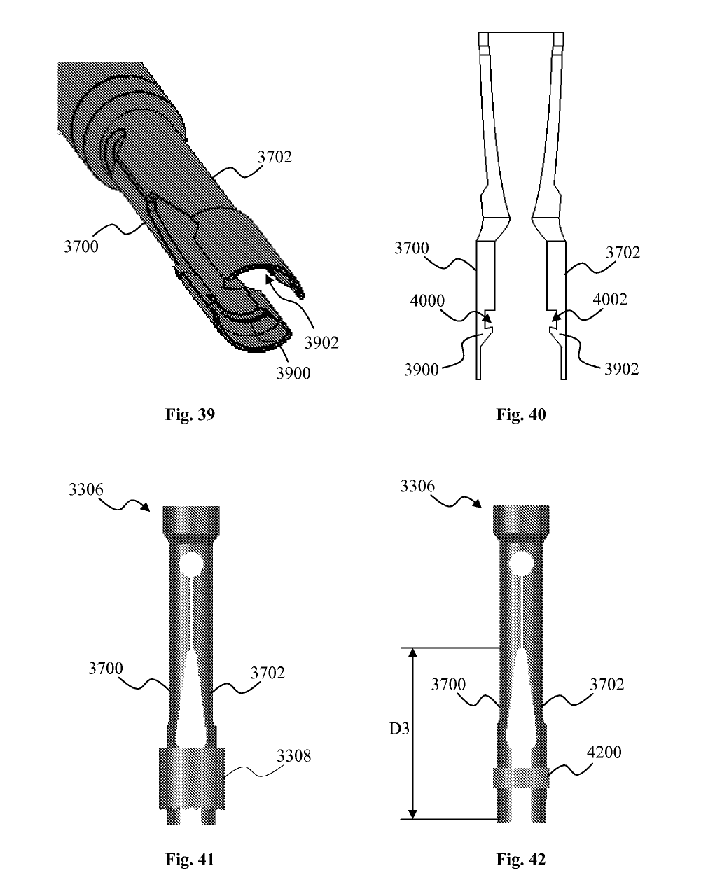

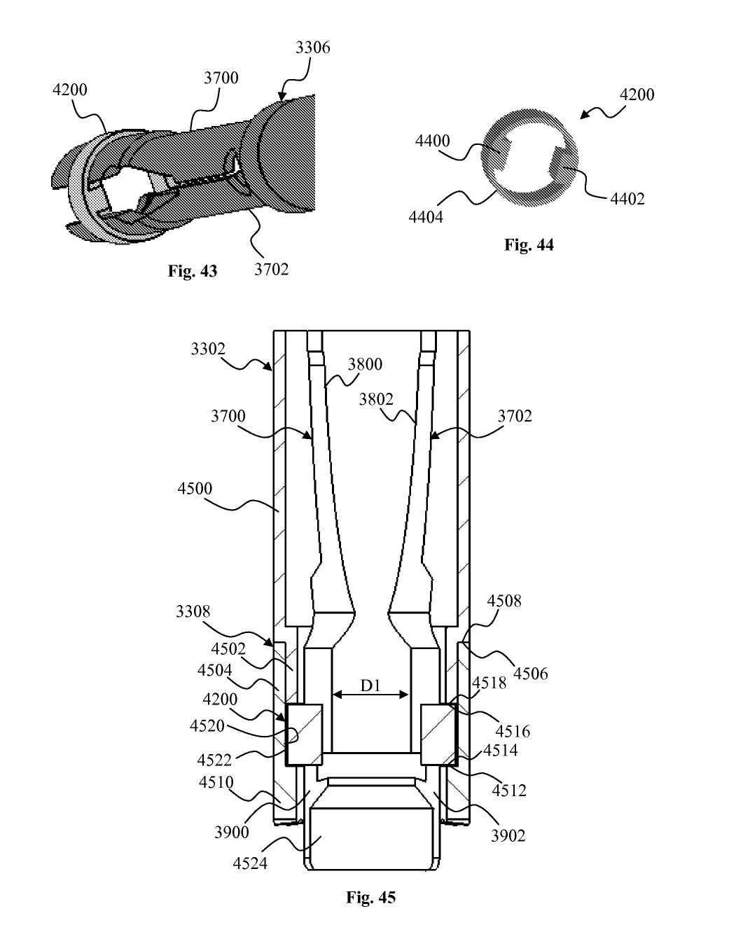

With additional reference to FIGS. 43 and 44, one embodiment of the ramp ring 4200 is illustrated that includes two protrusions 4400 and 4402 positioned within a ring 4404. As shown in FIG. 43, the protrusions 4400 and 4402 may be positioned between the fingers 3700 and 3702. In the present example, the protrusions 4400 and 4402 may have a width of approximately D1 (FIG. 38).

Referring to FIG. 45, a cross section of one embodiment of the distal end of the reduction device 3300 of FIG. 33 is illustrated. In the present example, a distal portion 4502 of the sleeve 3302 may have a smaller internal diameter than an adjacent portion 4500 of the sleeve. A proximal portion 4504 of the extension 3308 may have the same internal diameter as the adjacent portion 4500 of the sleeve 3302, and may slide over the distal portion 4502 of the sleeve with an upper surface 4506 of the extension abutting a surface 4508 of the sleeve where the sleeve narrows. A distal portion 4510 of the extension 3308 may have a smaller internal diameter than the proximal portion 4504 of the extension, thereby providing a shelf 4512 for receiving a lower surface 4514 of the ramp ring 4200. The shelf 4512 may limit downward movement of the ramp ring 4200. An upper surface 4516 of the ramp ring 4200 may abut a lower surface 4518 of the distal portion 4502 of the sleeve 3302. At least a portion of the external surface 4520 of the ramp ring 4200 may abut an interior surface 4522 of the proximal portion 4504 of the extension 3308.

In the present example, the fingers 3700 and 3702 may be coupled to a polyaxial head 4524 via the protrusions 3900 and 3902. A portion of the polyaxial head 4524 may be positioned within the indentions 4000 and 4002 (FIG. 40).

In operation, the ramp ring 4200 may be initially positioned at the proximal end of the opening 3704 (FIG. 37). When in this position, the protrusions 4400 and 4402 may engage the ramped sides 3800 and 3802, which may force the fingers 3700 and 3702 apart due to the relatively narrow distance separating the ramped sides at this point. After the distal ends of the fingers 3700 and 3702 are positioned over the polyaxial head 4524, the ramp ring 4200 may be moved downward toward the distal ends of the fingers in conjunction with the movement of the sleeve 3302. As the ramp ring 4200 moves downward, the protrusions 4400 and 4402 may be forced along the ramped sides 3800 and 3802, which are separated by an increasingly greater distance as they extend in the direction of the distal ends of the fingers 3700 and 3702. As the protrusions 4400 and 4402 move down the ramped sides 3800 and 3802 and into the wider portion of the opening 3704, they may no longer separate the fingers 3700 and 3702 and the fingers may be able to close around the polyaxial head 4524, thereby securing the coupling member 3306 to the polyaxial head.

Once the fingers 3700 and 3702 are coupled to the polyaxial head 4524, the ramp ring 4200 may be forced over the distal ends of the fingers by movement of the sleeve 3302. In this position, the ramp ring 4200 may lock the fingers 3700 and 3702 to the polyaxial head 4524.

To reverse the process, the sleeve 3302 may be moved upward, which forces the ramp ring 4200 into the narrower area between the fingers 3700 and 3702 (e.g., towards the point 3804 (FIG. 38). This forces the fingers 3700 and 3702 apart, thereby releasing the coupling member 3306 from the polyaxial head 4524.

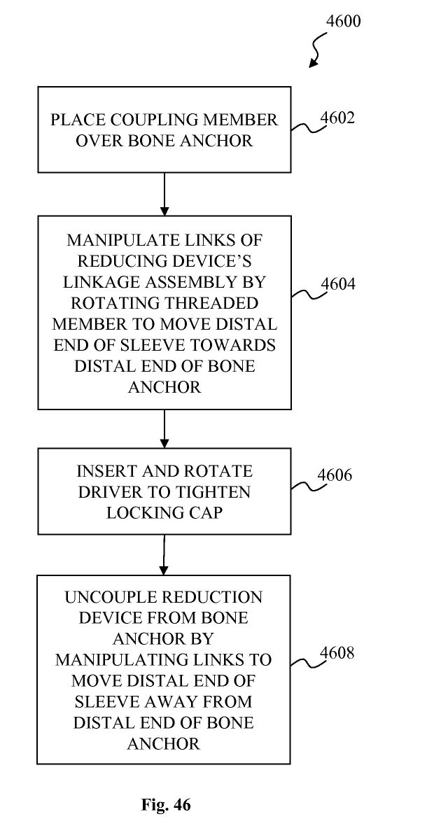

Referring to FIG. 46, a method 4600 illustrates one embodiment of a process using the reduction device 3300 of FIG. 33. In step 4602, the coupling member 3306 may be placed over a polyaxial head or other bone anchor system component. In step 4604, the sleeve 3302 may be moved in the direction of the polyaxial head by actuation of the linkage assembly 3304. As described above, this may force a ramp ring down fingers of the coupling member 3306, thereby locking the coupling member to the polyaxial head. Actuation of the linkage assembly 3304 may also force a rod into the polyaxial head, as has been described previously. In step 4606, a driver may be inserted into the reduction device 3300 to secure a locking cap to the polyaxial head. The reduction device 3300 may then be removed from the polyaxial head in step 4608 by reversing the direction of movement of the sleeve 3302, which releases the coupling member 3306 from the polyaxial head be forcing the ramp ring upwards.

It is understood that various materials may be used to form the reduction devices described in the present disclosure. For example, they may be made of one or more metals, plastics, or various combinations of materials. Furthermore, the reduction instruments may be formed with various dimensions to fit, for example, extensions of different sizes.

In addition, it is understood that terms such as “linkage assembly” are used for purposes of convenience and are not intended to be limiting. For example, the collar 1710 of FIG. 17 may be considered to be part of the linkage assembly in some embodiments, and not part of the linkage assembly in other embodiments. Similarly, the sleeve of various embodiments may include only the shell, or may include other components as described throughout the various embodiments. Accordingly, the descriptions of various parts of a reduction device as part of a linkage assembly or as part of another group of components are not intended to limit those parts to that particular group of components.

It is also understood that terms such as “above”, “below”, “top”, “bottom”, and “side” are relative and used for purposes of reference. As such, these and similar terms do not necessarily indicate an absolute position with respect to, for example, a surgical site.

Although only a few exemplary embodiments of this disclosure have been described in details above, those skilled in the art will readily appreciate that many modifications are possible in the exemplary embodiments without materially departing from the novel teachings and advantages of this disclosure. Also, features illustrated and discussed above with respect to some embodiments can be combined with features illustrated and discussed above with respect to other embodiments. Accordingly, all such modifications are intended to be included within the scope of this disclosure.

What is claimed is:

1. A spinal surgical device comprising:
a sleeve having a first proximal end, a first distal end configured to receive a rod, and a first longitudinal axis defining a first bore from the first proximal end to the first distal end;
a coupling member having a second proximal end, a second distal end configured to couple to a surgical device, and a second longitudinal axis defining a second bore from the second proximal end to the second distal end, wherein the coupling member is positioned at least partially within the sleeve with the first and second longitudinal axes oriented in substantially the same direction; and
a linkage assembly having a first link pivotally coupled to the sleeve and a first cross member, a second link pivotally coupled to the sleeve and a second cross member, a third link pivotally coupled to the coupling member and the first cross member, a fourth link pivotally coupled to the coupling member and the second cross member, and a threaded member coupling the first and second cross members, wherein a rotational position of the threaded member defines a distance between the first and second cross members.

2. The device of claim 1 wherein each of a plurality of pins coupling the first, second, third, and fourth links to the first and second cross members includes a longitudinal axis that is parallel to a longitudinal axis of the other pins of the plurality of pins.

3. The device of claim 1 further comprising a collar rotatably coupled to the coupling member at the second proximal end and positioned between the third and fourth links and the coupling member, wherein the third and fourth links are pivotally coupled to the collar.

4. The device of claim 3 wherein an interior surface of the collar facing the coupling member includes a groove configured to receive a flange formed on an exterior surface of the coupling member.

5. The device of claim 1 wherein the second distal end is threadably coupled to the surgical device.

6. The device of claim 1 wherein the threaded member includes an externally threaded first portion and a second portion having an internally threaded opening sized to engage the first portion.

7. The device of claim 6 wherein the first, second, third, and fourth links are configured to move the first proximal end along the first longitudinal axis in the direction of the first distal end as the first portion is advanced into the second portion.

8. The device of claim 1 wherein the first and second links are on substantially opposite sides of the first longitudinal axis.

9. The device of claim 8 wherein the third and fourth links are on substantially opposite sides of the first longitudinal axis.

10. The device of claim 1 wherein a longitudinal axis of the threaded member is substantially perpendicular to the first longitudinal axis.

11. The device of claim 1 wherein at least one of the first, second, third, and fourth links includes two support members.

12. The device of claim 1 wherein the coupling member includes first and second fingers positioned at the second distal end.

13. The device of claim 12 wherein the first finger includes a protrusion configured to engage a bone anchor.

14. The device of claim 12 further comprising a ramp ring having at least one protrusion, wherein the ramp ring is positioned adjacent to the first and second fingers with the protrusion positioned in an opening between the first and second fingers.

15. The device of claim 14 wherein the ramp ring is positioned outside of the first and second fingers and wherein the protrusion is on an interior surface of the ramp ring.

16. The device of claim 14 wherein the opening widens as it approaches the second distal end.

17. The device of claim 14 further comprising an extension positioned distally of the first distal end, wherein the ramp ring is positioned between at least a portion of the extension and the coupling member.

18. The device of claim 17 wherein the extension is coupled to the second distal end.

19. A spinal surgical device comprising:
a sleeve pivotally coupled to a distal end of first and second links positioned on substantially opposite sides of the sleeve;
a coupling member configured to couple to a surgical device and having a collar pivotally coupled to a distal end of opposing third and fourth links positioned on substantially opposite sides of the coupling member, wherein a longitudinal axis of the coupling member is substantially oriented with a longitudinal axis of the sleeve and wherein the coupling member is rotatable relative to the sleeve and collar; and
a threaded member pivotally coupled to a proximal end of the first, second, third, and fourth links, wherein a rotational position of the threaded member defines a position of a distal end of the coupling member relative to a distal end of the sleeve.

20. The device of claim 19 wherein a longitudinal axis of the threaded member is substantially perpendicular to the longitudinal axis of the coupling member.

21. The device of claim 19 further comprising a first cross member coupled to a first portion of the threaded member and pivotally coupled to the first and third links.

22. The device of claim 21 further comprising a second cross member coupled to a second portion of the threaded member and pivotally coupled to the third and fourth links.

23. The device of claim 19 wherein an angle between the first and third links and between the second and fourth links is capable of varying from about 45° to about 180°.

24. The device of claim 19 wherein longitudinal axes of first and second connectors coupling the first and second links to the sleeve are substantially parallel to longitudinal axes of third and fourth connectors coupling the third and fourth links to the collar.

25. The device of claim 24 wherein at least one of the first, second, third, and fourth connectors is a pin.

26. The device of claim 24 wherein at least one of the first, second, third, and fourth connectors includes a first portion and a second portion.

27. The device of claim 19 wherein the threaded member includes a first portion and a second portion configured to threadingly receive the first portion.

28. The device of claim 27 wherein the proximal ends of the first, second, third, and fourth links are configured to be a first distance from the sleeve when the first portion is fully received within the second portion, and to be a second distance from the sleeve when the first portion is fully retracted from the second portion, wherein the second distance is greater than the first distance.

29. A surgical system comprising:
a bone anchor coupled to a polyaxial head, wherein the polyaxial head includes first and second sidewalls forming a groove for receiving a rod;
an extension configured to removably couple to the polyaxial head; and
a reduction device having:
a sleeve sized to slide over the extension;
a coupling member having a distal end positioned at least partially within the sleeve and configured to couple to the extension; and
a linkage assembly including a threaded member, first and second links pivotally coupled to the sleeve and the threaded member, and third and fourth links pivotally coupled to the coupling member and the threaded member, wherein a rotational position of the threaded member defines a position of a distal portion of the sleeve relative to the polyaxial head.

30. A method for performing spine surgery, the method comprising:
inserting a distal end of a bone anchor into a vertebral body;
coupling an extension to a polyaxial head connected to a proximal end of the bone anchor;
sliding a sleeve of a reducing device over the extension;
coupling a coupling member of the reducing device to the extension within the sleeve, wherein the coupling member is coupled to the sleeve via a linkage assembly; and
moving a distal end of first and second links of the linkage assembly away from a distal end of third and fourth links of the linkage assembly by rotating a threaded member of the linkage assembly coupled to the first, second, third, and fourth links, wherein the moving alters a position of a distal end of the sleeve relative to the polyaxial head.

31. A spinal surgical device comprising:
a sleeve having a first proximal end, a first distal end configured to receive a rod, and a first longitudinal axis defining a first bore from the first proximal end to the first distal end;
a coupling member having a second proximal end, a second distal end configured to couple to a surgical device, and a second longitudinal axis defining a second bore from the second proximal end to the second distal end, wherein the coupling member is positioned at least partially within the sleeve with the first and second longitudinal axes oriented in substantially the same direction; and
a linkage assembly having first and second gears rotationally coupled to the sleeve, and first and second arms coupled to the first and second gears, respectively, wherein each of the first and second gears includes a plurality of teeth that engage a plurality of teeth on the coupling member, and wherein a position of the first and second arms defines a position of the coupling member relative to the sleeve.

32. A spinal surgical device comprising:
a sleeve having a first proximal end with a flange, a first distal end configured to receive a rod, and a first longitudinal axis defining a first bore from the first proximal end to the first distal end;
a handle having a second longitudinal axis defining a second bore through the handle, wherein the second bore includes a first threaded portion and a groove for receiving the flange of the sleeve;
a threaded boss member having a third proximal end, a third distal end configured to couple to a surgical device, and a third longitudinal axis defining a third bore from the third proximal end to the third distal end, wherein the third proximal end includes a lip extending around an opening to the third bore, and wherein the coupling member is positioned at least partially within the sleeve; and
a driver having a fourth proximal end, a fourth distal end, a fourth longitudinal axis oriented in substantially the same direction as the first longitudinal axis, and a flange configured to abut the lip and retain the fourth distal end in the third bore.

33. A method for performing spine surgery, the method comprising:
inserting a distal end of a bone anchor into a vertebral body;
placing a coupling member of a reducing device over a polyaxial head connected to a proximal end of the bone anchor;
moving a distal end of first and second links of the linkage assembly away from a distal end of third and fourth links of the linkage assembly by rotating a threaded member of the linkage assembly coupled to the first, second, third, and fourth links, wherein the moving couples the coupling member to the polyaxial head by altering a position of a distal end of the sleeve relative to a distal end of the coupling member.

34. The method of claim 33 wherein the moving further forces a rod into the polyaxial head.
